SEQUENCE LISTING (100 sequences)

SEQ ID NO: 1 - protein, 140 aa, synthetic construct
MDRLTSSFLLLIVPVYVLSQVTLKESGPGILQPSQTLSLTCSFSGFSLSTFGMGVSWIRQPSGKGLEWLAHIYWDDDKHYNPSLKSRLTISKDTSNNQVFLKITTVDTADTATYYCARRAWGDYDAFAYWGQGTLVTVSA

SEQ ID NO: 2 - protein, 136 aa, synthetic construct
MGRLTSSFLLLIVPAYVLSQVTLKESGPGILQPSQTLSLTCSFSGFSLSTYGIGVGWIRQPSGKGLEWLAHIWWNDNKYYNTALKSRLTISKDTSNNQVFLKIASVDTADTATYYCARSLPYFDYWGQGTTLTVSS

SEQ ID NO: 3 - protein, 140 aa, synthetic construct
MGWSCIIFFLVATATGVHSQVQLQQSGAELVRPGVSVKISCKGSGYTFTDYAMHWVKQSHAKSLEWIGIISTYYGNASYNQKFKGKATMTVDKSSSTAYMELARLTSEDSAIYYCARVPYYGSSYAMDYWGQGTSVTVSS

SEQ ID NO: 4 - protein, 134 aa, synthetic construct
MDFGLIFFIVALLKGVQCEVKLLESGGGLVQPGGSLKLSCAASGFDFSRYWMSWVRQAPGKGLEWIGEINPDSSTINYTPSLKDKFIISRDNAKNTLYLQMSKVRSEDTALYYCARYYSRFAYWGQGTLVTVSA

SEQ ID NO: 5 - protein, 110 aa, synthetic construct
EVQLVESGGGLVKPGGSRKLSCAASGFTLSDYGMHWVRQAPDKGLEWIAYISSDSNTIHYTDTVKGRFTISRDNARNTLFLQMTSLRSEDTAMYYCTRNYGMDYWGQGTS

SEQ ID NO: 6 - protein, 102 aa, synthetic construct
VLTQSPAIMSASLGERVTMTCTASSSVSSTYLHWYQQNPGSSPKLWIYSASNLASGVPTRFSGSGSGTSYSLTISSMEAEDAATYYCHQFHRSPYTFGGGTK

SEQ ID NO: 7 - protein, 131 aa, synthetic construct
MKLPVRLLVLMFWIPASSSDVLMTQTPLSLPVSLGDQASISCRSSQNIVHSDGNTYLEWYLQKPGQSPKLLIYKVSNRFSGVPDRFSGSGSGTDFTLKISRVEAEDLGVYYCFQGSHVPWTFGGGTKLEIK

SEQ ID NO: 8 - protein, 134 aa, synthetic construct; identical to SEQ ID NO: 4

SEQ ID NO: 9 - protein, 7 aa, synthetic construct
TFGMGVS

SEQ ID NO: 10 - protein, 16 aa, synthetic construct
HIYWDDDKHYNPSLKS

SEQ ID NO: 11 - protein, 11 aa, synthetic construct
RAWGDYDAFAY

SEQ ID NO: 12 - protein, 16 aa, synthetic construct
RSSQSLVHSNGNTYLH

SEQ ID NO: 13 - protein, 7 aa, synthetic construct
KVSNRFS

SEQ ID NO: 14 - protein, 9 aa, synthetic construct
SQSTHVPPT

SEQ ID NO: 15 - protein, 8 aa, synthetic construct
TYGIGVGW

SEQ ID NO: 16 - protein, 16 aa, synthetic construct
HIWWNDNKYYNTALKS

SEQ ID NO: 17 - protein, 7 aa, synthetic construct
SLPYFDY

SEQ ID NO: 18 - protein, 11 aa, synthetic construct
RASENIYSYLA

SEQ ID NO: 19 - protein, 7 aa, synthetic construct
NAKTLAE

SEQ ID NO: 20 - protein, 9 aa, synthetic construct
QHHYGIPRT

SEQ ID NO: 21 - protein, 6 aa, synthetic construct
DYAMHW

SEQ ID NO: 22 - protein, 17 aa, synthetic construct
IISTYYGNASYNQKFKG

SEQ ID NO: 23 - protein, 12 aa, synthetic construct
VPYYGSSYAMDY

SEQ ID NO: 24 - protein, 23 aa, synthetic construct
DVLMTQTPLSLPVSLGDQASISC

SEQ ID NO: 25 - protein, 7 aa, synthetic construct
KVSNRFS

SEQ ID NO: 26 - protein, 9 aa, synthetic construct
FQGSHVPWT

SEQ ID NO: 27 - protein, 5 aa, synthetic construct
RYWMS

SEQ ID NO: 28 - protein, 17 aa, synthetic construct
EINPDSSTINYTPSLKD

SEQ ID NO: 29 - protein, 7 aa, synthetic construct
YYSRFAY

SEQ ID NO: 30 - protein, 14 aa, synthetic construct
RSSTGAVTTRNYAN

SEQ ID NO: 31 - protein, 7 aa, synthetic construct
DTNNRAP

SEQ ID NO: 32 - protein, 9 aa, synthetic construct
ALWYSNHWV

SEQ ID NO: 33 - DNA, 368 bp, synthetic construct
  source 1..368: mol_type = other DNA
tatcccaggttactctgaaagagtctggccctggtatattgcagccctcccagaccctcagtctgacctgttctttctctgggttttcactgagcacttttggtatgggtgtgagctggattcgtcagccttcagggaagggtctggagtggctggcacacatttattgggatgatgacaagcactataacccatccttgaagagccggctcacaatctccaaggatacctccaacaaccaggtattcctcaagatcaccactgtggacactgcagatactgccacatactactgtgctcgaagagcttggggtgattacgacgcgtttgcttactggggccaagggactctggtcactgtctctgca

SEQ ID NO: 34 - DNA, 367 bp, synthetic construct
  source 1..367: mol_type = other DNA
tgcatatgtcctctcccaggttactctgaaagagtctggccctgggatattgcagccctcccagaccctcagtctgacttgttctttctctgggttttcactgagcacttatggtataggagtaggctggattcgtcagccttcagggaagggtctggagtggctggcacacatttggtggaatgataataagtactataacacagccctgaagagccggctcacaatctccaaggatacctccaacaaccaggtattcctcaagatcgccagtgtggacactgcagatactgccacatactactgtgctcgctcattaccctactttgactactggggccaaggcaccactctcacagtctcctca

SEQ ID NO: 35 - DNA, 363 bp, synthetic construct
  source 1..363: mol_type = other DNA
caggtccagctgcagcagtctggggctgagctggtgaggcctggggtctcagtgaagatttcctgcaagggttctggctacacattcactgattatgctatgcactgggtgaagcagagtcatgcaaagagtctagagtggattggaattattagtacttactatggtaatgctagctacaaccagaagttcaagggcaaggccacaatgactgtagacaaatcgtccagcacagcctatatggaacttgccagactgacatctgaggattctgccatctattactgtgcaagagtcccttactacggtagtagctatgctatggactactggggtcaaggaacctcagtcaccgtctcctca

SEQ ID NO: 36 - DNA, 348 bp, synthetic construct
  source 1..348: mol_type = other DNA
gaggtgaagcttctcgagtctggaggtggcctggtgcagcctggaggatccctgaaactctcctgtgcagcctcaggattcgattttagtagatactggatgagttgggtccggcaggctccagggaaagggctagaatggattggagaaattaatccagatagcagtacgataaactatacgccatctctaaaggataaattcatcatctccagagacaacgccaaaaatacgctgtacctgcaaatgagcaaagtgagatctgaggacacagccctttattactgtgcaagatactattcccgttttgcttactggggccaagggactctggtcactgtctctgca

SEQ ID NO: 37 - DNA, 336 bp, synthetic construct
  source 1..336: mol_type = other DNA
gatgttgtgatgacccaaactccactctccctgcctgtcagtcttggagatcaagcctccatctcttgcagatctagtcagagccttgtacacagtaatggaaacacctatttacattggtacctgcagaagccaggccagtctccaaagctcctgatctacaaagtttccaaccgattttctggggtcccagacaggttcagtggcagtggatcagggacagatttcacactcaagatcagcagagtggaggctgaggatctgggagtttatttctgctctcaaagtacacatgttcctccgacgttcggtggaggcaccaagctggaaatcaaa

SEQ ID NO: 38 - DNA, 321 bp, synthetic construct
  source 1..321: mol_type = other DNA
gacatccagatgactcagtctccagcctccctatctgcatctgtgggagaaactgtcaccatcacatgtcgagcaagtgagaatatttacagttatttagcatggtatcagcagaaacagggaaaatctcctcagttcctggtctataatgcaaaaaccttagcagaaggtgtgccatcaaggttcagtggcagtggatcaggcacacagttttctctgaagatcaacagcctgcagcctgaagattttgggagttattactgtcaacatcattatggtattcctcggacgttcggtggaggcaccaagttggaaatcaaa

SEQ ID NO: 39 - DNA, 336 bp, synthetic construct
  source 1..336: mol_type = other DNA
gatgttttgatgacccaaactccactctccctgcctgtcagtcttggagatcaagcctccatctcttgcagatctagtcagaacattgtacatagtgatggaaacacctatttagaatggtacctgcagaaaccaggccagtctccaaagctcctgatctacaaagtttccaaccgattttctggggtcccagacaggttcagtggcagtggatcagggacagatttcacactcaagatcagcagagtggaggctgaggatctgggagtttattactgctttcaaggttcacatgttccgtggacgttcggtggaggcaccaagctggaaatcaaa

SEQ ID NO: 40 - DNA, 326 bp, synthetic construct
  source 1..326: mol_type = other DNA
aggctgttgtgactcaggaatctgcactcaccacatcacctggtgaaacagtcacactcacttgtcgctcaagtactggggctgttacaactcgtaactatgccaactgggtccaagaaaaaccagatcatttattcactggtctaataggtgataccaacaaccgagctccaggtgttcctgccagattctcaggctccctgattggagacaaggctgccctcaccatcacaggggcacagactgaggatgaggcaatatatttctgtgctctatggtacagcaaccattgggtgttcggtggaggaaccaaactgactgtccta

SEQ ID NO: 41 - DNA, 21 bp, synthetic construct
  source 1..21: mol_type = other DNA
acttttggtatgggtgtgagc

SEQ ID NO: 42 - DNA, 48 bp, synthetic construct
  source 1..48: mol_type = other DNA
cacatttattgggatgatgacaagcactataacccatccttgaagagc

SEQ ID NO: 43 - DNA, 33 bp, synthetic construct
  source 1..33: mol_type = other DNA
agagcttggggtgattacgacgcgtttgcttac

SEQ ID NO: 44 - DNA, 48 bp, synthetic construct
  source 1..48: mol_type = other DNA
agatctagtcagagccttgtacacagtaatggaaacacctatttacat

SEQ ID NO: 45 - DNA, 21 bp, synthetic construct
  source 1..21: mol_type = other DNA
aaagtttccaaccgattttct

SEQ ID NO: 46 - DNA, 31 bp, synthetic construct
  source 1..31: mol_type = other DNA
tctcaaagtacacatgttcctccgacgttcg

SEQ ID NO: 47 - DNA, 21 bp, synthetic construct
  source 1..21: mol_type = other DNA
acttatggtataggagtaggc

SEQ ID NO: 48 - DNA, 48 bp, synthetic construct
  source 1..48: mol_type = other DNA
cacatttggtggaatgataataagtactataacacagccctgaagagc

SEQ ID NO: 49 - DNA, 21 bp, synthetic construct
  source 1..21: mol_type = other DNA
tcattaccctactttgactac

SEQ ID NO: 50 - DNA, 33 bp, synthetic construct
  source 1..33: mol_type = other DNA
cgagcaagtgagaatatttacagttatttagca

SEQ ID NO: 51 - DNA, 21 bp, synthetic construct
  source 1..21: mol_type = other DNA
aatgcaaaaaccttagcagaa

SEQ ID NO: 52 - DNA, 27 bp, synthetic construct
  source 1..27: mol_type = other DNA
caacatcattatggtattcctcggacg

SEQ ID NO: 53 - DNA, 15 bp, synthetic construct
  source 1..15: mol_type = other DNA
gattatgctatgcac

SEQ ID NO: 54 - DNA, 48 bp, synthetic construct
  source 1..48: mol_type = other DNA
attagtacttactatggtaatgctagctacaaccagaagttcaagggc

SEQ ID NO: 55 - DNA, 36 bp, synthetic construct
  source 1..36: mol_type = other DNA
gtcccttactacggtagtagctatgctatggactac

SEQ ID NO: 56 - DNA, 34 bp, synthetic construct
  source 1..34: mol_type = other DNA
agatctagtcagaacattgtacatagtgatggaa

SEQ ID NO: 57 - DNA, 21 bp, synthetic construct
  source 1..21: mol_type = other DNA
aaagtttccaaccgattttct

SEQ ID NO: 58 - DNA, 27 bp, synthetic construct
  source 1..27: mol_type = other DNA
tttcaaggttcacatgttccgtggacg

SEQ ID NO: 59 - DNA, 15 bp, synthetic construct
  source 1..15: mol_type = other DNA
agatactggatgagt

SEQ ID NO: 60 - DNA, 51 bp, synthetic construct
  source 1..51: mol_type = other DNA
gaaattaatccagatagcagtacgataaactatacgccatctctaaaggat

SEQ ID NO: 61 - DNA, 21 bp, synthetic construct
  source 1..21: mol_type = other DNA
tactattcccgttttgcttac

SEQ ID NO: 62 - DNA, 42 bp, synthetic construct
  source 1..42: mol_type = other DNA
cgctcaagtactggggctgttacaactcgtaactatgccaac

SEQ ID NO: 63 - DNA, 21 bp, synthetic construct
  source 1..21: mol_type = other DNA
gataccaacaaccgagctcca

SEQ ID NO: 64 - DNA, 27 bp, synthetic construct
  source 1..27: mol_type = other DNA
gctctatggtacagcaaccattgggtg

SEQ ID NO: 65 - protein, 19 aa, synthetic construct
MDRLTSSFLLLIVPVYVLS

SEQ ID NO: 66 - protein, 19 aa, synthetic construct
MKLPVRLLVLMFWIPASSS

SEQ ID NO: 67 - protein, 19 aa, synthetic construct
MGRLTSSFLLLIVPAYVLS

SEQ ID NO: 68 - protein, 20 aa, synthetic construct
MSVPTQVLGLLLLWLTGARC

SEQ ID NO: 69 - protein, 19 aa, synthetic construct
MGWSCIIFFLVATATGVHS

SEQ ID NO: 70 - protein, 18 aa, synthetic construct
MDFGLIFFIVALLKGVQC

SEQ ID NO: 71 - protein, 19 aa, synthetic construct
MAWISLILSLLALSSGAIS

SEQ ID NO: 72 - DNA, 52 bp, synthetic construct
  source 1..52: mol_type = other DNA
atggacaggcttacttcctcattcctactgctgattgtccctgtctatgtcc

SEQ ID NO: 73 - DNA, 57 bp, synthetic construct
  source 1..57: mol_type = other DNA
atgaagttgcctgttaggctgttggtgctgatgttctggattcctgcttccagcagt

SEQ ID NO: 74 - DNA, 41 bp, synthetic construct
  source 1..41: mol_type = other DNA
atgggcaggcttacttcttcattcctgctactgattgttcc

SEQ ID NO: 75 - DNA, 60 bp, synthetic construct
  source 1..60: mol_type = other DNA
atgagtgtgcccactcaggtcctggggttgctgctgctgtggcttacaggtgccagatgt

SEQ ID NO: 76 - DNA, 57 bp, synthetic construct
  source 1..57: mol_type = other DNA
atgggttggagctgtatcatcttctttctggtagcaacagctacaggtgtgcactcc

SEQ ID NO: 77 - DNA, 54 bp, synthetic construct
  source 1..54: mol_type = other DNA
atggattttgggctgattttttttattgttgctcttttaaaaggggtccagtgt

SEQ ID NO: 78 - DNA, 57 bp, synthetic construct
  source 1..57: mol_type = other DNA
atggcctggatttcacttatactctctctcctggctctcagctcaggggccatttcc

SEQ ID NO: 79 - protein, 161 aa, synthetic construct
MFVQIAASLAAASSIALGIPGAATPIDESQLPVGPQVSVTDSAQHTGPFAASSPLTITVKPGAPCVRADGYQESMVTRVLDDKGHQVWTGTFDESKLIGGTGLGTATFHVGSPAAAFNFHGSERTTYRTLSYCAYPHYVNGTRERLSQVSVKTFMVDPALN

SEQ ID NO: 80 - protein, 161 aa, synthetic construct
MFVQIAASLAAASSIALGMPGAATPIDESQLPVGPQVSVTDSAQHTGPFAASSPLAITVKPGAPCVRADGYQESMVTRVLDDKGHQVWTGTFDESKLIGGTGLGTATFHVESPAAAFNFHGNERTTYRTLSYCAYPHYVNGTRERLSQVSVKTFMVDPALN

SEQ ID NO: 81 - protein, 161 aa, synthetic construct
MFVQIAASLAAASSIALGIPGAATPIDESQLPVGPQVSVTDSAQHTGPFAASNPLTITVKPGAPCVRADGYQESMVTRVLDDKGHQVWTGTFDESKLIGGTGLGTATFHVESPAAAFNFHGNERTTYRTLSYCAYPHYVNGTRERLSQVSVKTFMVDPALN

SEQ ID NO: 82 - protein, 161 aa, synthetic construct
MFVQIAASLAAASSIALGIPGAATPIDESQLPVGPQVSVTDSAQHTGPFAASSPLTITVKPGAPCVRADGRQQSLVARVLDDKGHQVWSGTFDESQLIGGTGRGTATFHVGSAAAAFNFHGGKRTTYRALGYCAYPHYVNGTRERLSQVSVKTFTVDPAIN

SEQ ID NO: 83 - protein, 161 aa, synthetic construct
MFVQIAASLAAASTIALGVPGAATPLNGSQLPTGPQVSVTDPAQHTGPFSTATPLTIKVKPSAPCVRADGRQQSLVARVLDDKGHQVWSGTFDESQLIGGTGRGTATFHVGSAAAAFNFHGGKRTTYRALGYCAYPHYVNGTRERLSQVSVKTFTVDPAIN

SEQ ID NO: 84 - protein, 161 aa, synthetic construct
MFVQIAASLAAASTIALGVPGAATPLNGSQLPTDPRVSVTDPAQHTGPFSTATPLTIKVKPSAPCVRADGRQQSLVARVLDDKGHQVWSGTFDESQLIGGTGRGTATFHVGSAAAAFNFHGGKRTTYRALGYCAYPHYVNGTRERLSQVSVKTFTVDPAIN

SEQ ID NO: 85 - protein, 161 aa, synthetic construct
MFVQIAASLAAASTLALGAPGAPTPIDESHLPVGPQVSVTDSAQHTGPFTATTPLTIKVKPNAPCVRADGYQASLVARVLDGKGRQVWTGTFDESQLIGGKGFGTATFHVDSAAVAFNYHGDKRTTYRAVSYCAYPHYVNGARERLSQVSAKTFAVNPAAN

SEQ ID NO: 86 - protein, 161 aa, synthetic construct
MFVQIAASLAAASTLALGAPGAPTPIDESHLPVGPQVSVTDSAQHTGPFTATTPLTIKVKPNAPCVRADGYQASLVARVLDGKGRQVWTGTFDESQLIGGKGFGTATFHVDSAAAAFNYHGDKRTTYRAVSYCAYPHYVNGARERLSQVSAKTFAVNPAAN

SEQ ID NO: 87 - protein, 161 aa, synthetic construct
MFVQIAASLAAASSIALGIPGAATPIDESQLPVGPQVSVTDFAQHTGPFAASSPLPITVKPGAPCVRADGYQESMVTRVLDDKGHQVWTGTFDESKLIGGTGLGTATFHVGSPAAAFNFHGSERTTYRTLSYCAYPHYVNGTRERLSQVSVKTFMVDPALN

SEQ ID NO: 88 - protein, 161 aa, synthetic construct
MFVQIAASLAAASSIALGIPGAATPIDESQLPVGPQVSVTDSAQHTGPFAASSPLTITVKPGAPCVRADGYQESMVTRVLDDKGHQVWTGAFDESKLIGGTGLGTATFHVGSPAAAFNFHGSERTTYRTLSYCAYPHYVNGTRERLSQVSVKTFMVDPALN

SEQ ID NO: 89 - protein, 161 aa, synthetic construct
MLIQIAASIAAASAMTFGIPGTVTPINTNHLSPEPQVSVTDISQHTGPFAVTTPLTVKVTPSAPCVSSDGHQESIVARILDDQGHQVWAGTFDESTLIGGTGRGTATFHVGSAAATFNYHGSQRTTYRTLGYCAYPHYVDGNRERLSKVGVAPFTVDPAKN

SEQ ID NO: 90 - protein, 161 aa, synthetic construct; identical to SEQ ID NO: 89

SEQ ID NO: 91 - protein, 168 aa, synthetic construct
MTYWGITMFVQIAASLAAASTLALGAPGAPTPIDESHLPVGPQVSVTDSAQHTGPFTATTPLTIKVKPNAPCVRADGYQASLVARVLDGKGRQVWTGTFDESQLIGGKGFGTATFHVDSAAVAFNYHGDKRTTYRAVSYCAYPHYVNGARERLSQVSAKTFAVNPAAN

SEQ ID NO: 92 - protein, 161 aa, synthetic construct
MFVQIAASLAAASSIALGIPGAATPIDESQLPVGPQVSVTDSAQHTGPFAASSPLTITVKPGAPCVRADGYQESMVTRVLDDKGHQVWTGTFDESKLIGGTGLGTATFHVGSPAAAFNFHGSERTTYRTLSYCAYSHYVNGTRERLSQVSVKTFMVDPALN

SEQ ID NO: 93 - protein, 161 aa, synthetic construct
MFVQIAASLAAASSIALGIPGAATPIDESQLPVGPQVSVTDSAQHTGPFAASSPLAITVKPGAPCVRADGYQESMVTRVLDDKGHQVWTGTFDESKLIGGTGLGTATFHVESPAAAFNFHGNERTTYRTLSYCAYPHYVNGTRERLSQVSVKTFMVDPALN

SEQ ID NO: 94 - protein, 161 aa, synthetic construct
MFVQIAASLAAASSIALGIPGAATPIDESQLPVGPQVSVTDFAQHTGPFAASSPLTITVKPGAPCVRADGYQESMVTRVLDDKGHQVWTGTFDESKLIGGTGLGTATFHVGSPAAAFNFHGSERTTYRTLSYCAYPHYVNGTRERLSQVSVKTFMVDPALN

SEQ ID NO: 95 - protein, 161 aa, synthetic construct
MFVQIAASLAAASSIALGIPGAATPIDESQLPVGPQVSVTDSAQHTGPFAASSPLIITVKPGAPCVRADGYQESMVTRVLDDKGHQVWTGTFDESKLIGGTGLGTATFHVGSPAAAFNFHGSERTTYRTLSYCAYPHYVNGTRERLSQVSVKTFMVDPALN

SEQ ID NO: 96 - protein, 161 aa, synthetic construct
MFVQIAASLAAASSIALGIPGAATPIDESQLPVGPQVSVTDSAQHTGPFAASSPLTITVKPGAPCVRADGYQQSMVTRVLDDKGHQVWTGTFDESKLIGGTGLGTATFHVGSPAAAFNFHGSERTTYRTLSYCAYPHYVNGTRERLSQVSVKTFMVDPALN

SEQ ID NO: 97 - protein, 161 aa, synthetic construct
MFVQIAASLAAASSIALGIPGAATPIDESQLPVGPQVSVTDSAQHTGPFAASSPLTITVKPGAPCVRADGYQESMVTRVLDDKGHQVWTGTFDESKLIGGTGLGTATFHVGSPAAAFNFHGSERTTYRTLSYCAYPHYVNGTRERLSQVSVKTFMVDPTLN

SEQ ID NO: 98 - protein, 161 aa, synthetic construct
MFVQIAASLAAASSIALGIPGAATPIDESQLPVGPQVSVTDSAQHTGPFAASSPLTITVKPGAPCVRADGYQESMVTRVLDDKGHQVWTGTFDESKLIGGTGLGTATFHVESPAAAFNFHGSERTTYRTLSYCAYPHYVNGTRERLSQVSVKTFMVDPALN

SEQ ID NO: 99 - protein, 161 aa, synthetic construct
MFVQIAASLAAASTIALGVPGAATPLNGSQLPTGPQVSVTDPAQHTGPFSTATPLTITVKPSAPCVRADGRQQSLVARVLDDKGHQVWSGTFDESQLIGGTGRGTATFHVGSAAAAFNFHGGKRTTYRALGYCAYPHYVNGTRERLSQVSVKTFTVDPAIN

SEQ ID NO: 100 - protein, 42 aa, synthetic construct
MKYLLPTAAAGLLLLAAQPAMAMDHHHHHHHHGGSENLYFQG

(19) United States
(12) Patent Application Publication
(10) Pub. No.: US 2024/0400656 A1
(43) Pub. Date: Dec. 5, 2024

(54) ANTI-ROXP ANTIBODIES AND USES THEREOF

(71) Applicant: Washington University, St. Louis, MO (US)

(72) Inventors: William McCoy, IV, St. Louis, MO (US); Maxenia Ilagan, St. Louis, MO (US); Alexander Aleem, St. Louis, MO (US)

(21) Appl. No.: 18/732,214

(22) Filed: Jun. 3, 2024

Related U.S. Application Data
(60) Provisional application No. 63/470,375, filed on Jun. 1, 2023.

(51) Int. Cl. C07K 16/12; G01N 33/569

(52) U.S. Cl. CPC C07K 16/1267; G01N 33/56911; C07K 2317/565; G01N 2333/195; G01N 2469/10; G01N 2470/06

(57) ABSTRACT

Disclosures herein are directed to antibodies and epitope binding agents that specifically bind to RoxP of Cutebacterium sp. and compositions thereof for use in clinical and non-clinical applications.

### ACKNOWLEDGEMENT OF GOVERNMENT SUPPORT

This invention was made with government support under AR076464, TR002345, and TR002346 awarded by the National Institutes of Health. The government has certain rights in the invention.

### FIELD OF THE INVENTION

The present invention relates to monoclonal antibodies against RoxP and method of using these antibodies for diagnosis of Cutibacterium related infections.

### INCORPORATION OF SEQUENCE LISTING

The present application contains a Sequence Listing which has been submitted in .XML format via Patent Center and is hereby incorporated by reference in its entirety. Said WIPO Sequence Listing was created on May 31, 2024, is named 047563_803330_Sequence_Listing. and is 94 kilobytes in size

### BACKGROUND

Each year, over a million infections related to indwelling medical devices take place in the USA, leading to notable morbidity and mortality. Most of these infections are due to microbes that are commensal to humans. Cutibacterium acnes is a slow growing bacterium, and predominantly residues within follicles and pores on the skin. It is considered to be an opportunistic pathogen causing a range of postoperative and device-related infections e.g., surgical infections, post-neurosurgical infections, infected joint prostheses, neurosurgical shunt infections and endocarditis in patients with prosthetic heart valves. C. acnes may also play a role in other conditions, including SAPHO (synovitis, acne, pustulosis, hyperostosis, osteitis) syndrome, sarcoidosis and sciatica. It is a common contaminant in blood and cerebrospinal fluid cultures.

Infections by C. acnes ae treatable by antibiotics but delayed identification of C. acnes infections due to the requirement of an extended anaerobic cultivation can create a roadblock for timely medical intervention. Thus, strategies to identify C. acnes infections are needed.

### SUMMARY OF THE INVENTION

In an aspect, the current disclosure encompasses an isolated antibody, epitope binding fragment or variant thereof that binds to Rox-P, comprising (a) an immunoglobulin heavy chain variable region (VH) at least 90% identical to an amino acid sequence set forth in one of SEQ ID NOS: 1-4; or (b) an immunoglobulin light chain variable region (VL) at least 90% identical to an amino acid sequence set forth in one of SEQ ID NOS: 5-8; or both (a) and (b) above.

In some aspects, the isolated antibody, epitope binding fragment or variant thereof comprises one or more of: a HC-CDR1 comprising an amino acid sequence at least 80% identical to the amino acid sequence of SEQ ID NO: 9, 15, 21, or 27, a HC-CDR2 comprising an amino acid sequence at least 80% identical to the amino acid sequence of SEQ ID NO: 10, 16, 22, or 28, a HC-CDR3 comprising an amino acid sequence at least 80% identical to the amino acid sequence of SEQ ID NO: 11, 17, 23, or 29, a LC-CDR1 comprising an amino acid sequence at least 80% identical to the amino acid sequence of SEQ ID NO: 12, 18, 24, or 30, a LC-CDR2 comprising an amino acid sequence at least 80% identical to the amino acid sequence of SEQ ID NO: 13, 19, 25, or 31; or a LC-CDR3 comprising an amino acid sequence at least 80% identical to the amino acid sequence of SEQ ID NO: 14, 20, 26, or 32. In some aspects, the VH comprises an amino acid sequence at least 90% identical to the amino acid sequence set forth in any one of SEQ ID NOs: 1-4. In some aspects, the VL comprises an amino acid sequence at least 90% identical to the amino acid sequence set forth in any one of SEQ ID NO: 5-8. In some aspects, the antibody comprises: a heavy chain variable region having the sequence of SEQ ID NO. 1 and a light chain variable region having the sequence of SEQ ID NO. 5; a heavy chain variable region having the sequence of SEQ ID NO. 1 and a light chain variable region having the sequence of SEQ ID NO. 6; a heavy chain variable region having the sequence of SEQ ID NO. 1 and a light chain variable region having the sequence of SEQ ID NO. 7; a heavy chain variable region having the sequence of SEQ ID NO. 1 and a light chain variable region having the sequence of SEQ ID NO. 8; a heavy chain variable region having the sequence of SEQ ID NO. 2 and a light chain variable region having the sequence of SEQ ID NO. 5; a heavy chain variable region having the sequence of SEQ ID NO. 2 and a light chain variable region having the sequence of SEQ ID NO. 6; a heavy chain variable region having the sequence of SEQ ID NO. 2 and a light chain variable region having the sequence of SEQ ID NO. 7; a heavy chain variable region having the sequence of SEQ ID NO. 2 and a light chain variable region having the sequence of SEQ ID NO. 8; a heavy chain variable region having the sequence of SEQ ID NO. 3 and a light chain variable region having the sequence of SEQ ID NO. 5; a heavy chain variable region having the sequence of SEQ ID NO. 3 and a light chain variable region having the sequence of SEQ ID NO. 6; a heavy chain variable region having the sequence of SEQ ID NO. 3 and a light chain variable region having the sequence of SEQ ID NO. 7; a heavy chain variable region having the sequence of SEQ ID NO. 3 and a light chain variable region having the sequence of SEQ ID NO. 8; a heavy chain variable region having the sequence of SEQ ID NO. 4 and a light chain variable region having the sequence of SEQ ID NO. 5; a heavy chain variable region having the sequence of SEQ ID NO. 4 and a light chain variable region having the sequence of SEQ ID NO. 6; a heavy chain variable region having the sequence of SEQ ID NO. 5 and a light chain variable region having the sequence of SEQ ID NO. 7; or a heavy chain variable region having the sequence of SEQ ID NO. 6 and a light chain variable region having the sequence of SEQ ID NO. 8.

In some aspects, the isolated antibody is a full-length antibody. In some aspects, the antibody is any one of a monoclonal antibody, an IgG, Fv, single chain antibody, nanobody, diabody, scFv, Fab, F(ab′) 2, and Fab. In some aspects, the antibody further comprises a signal sequence. In some aspects, the antibody further comprises a detection molecule. In some aspects, the detection molecule comprises a fluorescent label, phosphorescent molecule, chemiluminescent molecule, radioactive isotope, chromophore, luminescent molecule, photoaffinity molecule, colored particle and/or ligand, such as a biotin fluorescent dye, electrochemiluminescense dye, metal-chelate complex, or labels.

In some aspects, the current disclosure also encompasses a polynucleotide sequence encoding an isolated antibody, epitope binding fragment or variant thereof as disclosed herein. In some aspects, the polynucleotide comprises a nucleic acid sequence at least 80% identical to: a nHC-CDR1 corresponding to any one of SEQ ID NO: 41, 47, 53, or 59; a nHC-CDR2 corresponding to any one of SEQ ID NO: 42, 48, 54, or 60; a nHC-CDR3 corresponding to any one of SEQ ID NO: 43, 49, 55, or 61; a nLC-CDR1 corresponding to any one of SEQ ID NO: 44, 50, 56, or 62; a nLC-CDR2 corresponding to any one of SEQ ID NO: 45, 51, 57, or 63; or a nLC-CDR3 corresponding to any one of SEQ ID NO: 46, 52, 58, or 64. In some aspects, the polynucleotide encodes a variable heavy chain (VH) comprising a nucleic acid sequence at least 90% identical to any one of SEQ ID NO. 33-36. In some aspects, the variable light chain (VL) comprises a nucleic acid sequence at least 80% identical to one of SEQ ID NO. 37-40.

In some aspects, the current disclosure also encompasses a host cell comprising the polynucleotide as disclosed herein. In some aspects, the host cell comprises a first nucleic acid encoding any one of the variable heavy chain (VH) polypeptide as disclosed herein; and/or a second nucleic acid encoding any one of the variable light chain (VL) polypeptide as disclosed herein. In some aspects, the host cell is a splenic B lymphocyte or a hybridoma.

In some aspects, the current disclosure also encompasses a method of isolating the antibody, epitope binding fragment or variant thereof as disclosed herein, comprising culturing a host cell as disclosed herein under conditions suitable for the host cell to express the antibody, epitope binding fragment, or variant thereof and purifying the antibody, epitope binding fragment, or variant thereof from the cell.

In some aspects, the current disclosure also encompasses a method of detecting Cutibacterium sp. in a sample, the method comprising: contacting the sample with the isolated antibody, epitope binding fragment or variant thereof as disclosed; wherein the isolated antibody, epitope binding fragment or variant thereof specifically binds to RoxP to form an antibody-RoxP complex; using a detector to determine presence of the antibody-RoxP complex; quantifying the levels of RoxP in the antibody-RoxP complex; and determining the amount of Cutibacterium sp in the sample based on the quantity of RoxP in the sample.

In some aspects of the method, the isolated antibody, epitope binding fragment or variant thereof further comprises a detection molecule. In some aspects, the method further comprises contacting the antibody-RoxP complex with a reporter molecule that specifically binds to the isolated antibody, epitope binding fragment or variant thereof and/or the antibody-RoxP complex, wherein the reporter molecule comprises a detector molecule. In some aspects, the reporter molecule is a secondary antibody. In some aspects, the detector molecule is selected from a fluorescent label, a phosphorescent molecule, a chemiluminescent molecule, a radioactive isotope, a chromophore, a luminescent molecule, a photoaffinity molecule, colored particles and/or ligands. In some aspects, the detector is selected from a microscope, a FACS, fluorescent detector, chemiluminescent detector, autoradiography equipment, and a scintillation counter. In some aspects, the sample is a biological sample from a subject. In some aspects, the biological sample is selected from a biological fluid, tissue, and cell culture. In some aspects, the biological fluid is selected from CSF, blood, saliva, tears, and pus.

In some aspects, the current disclosure also encompasses a method of quantifying RoxP in a sample, the method comprising: contacting the sample with the isolated antibody, epitope binding fragment or variant thereof as disclosed herein; wherein the isolated antibody, epitope binding fragment or variant thereof specifically binds to RoxP to form an antibody-RoxP complex; using a detector to determine presence of the antibody-RoxP complex; and quantifying the levels of RoxP in the antibody-RoxP complex, wherein the quantified levels are indicative of the quantity of RoxP in the sample.

In some aspects, the current disclosure encompasses a method of treating a subject in need thereof, the method comprising administering a therapeutically effective amount of a composition comprising antibody, epitope binding fragment, or variant thereof disclosed herein to a subject. In some aspects, the subject has or is likely to have a Cutibacterium infection.

In some aspects, the current disclosure encompasses a kit comprising: (a) the isolated antibody, epitope binding fragment or variant thereof as disclosed herein; (b) optionally a reporter molecule that binds to (a); (c) reagents; and (d) instructions for use. In some aspects, the current disclosure also encompasses a kit for ELISA and related applications comprising: (a) the isolated antibody, epitope binding fragment or variant thereof as disclosed herein; (b) reagents; and (c) instructions for use.

### BRIEF DESCRIPTION OF THE DRAWINGS

The patent or application file contains at least one drawing executed in color. Copies of this patent or patent application with color drawing(s) will be provided by the Office by request and payment of the necessary fee.

Embodiments of the present inventive concept are illustrated by way of example in which like reference numerals indicate similar elements and in which:

FIG. 1A shows indirect ELISA curves showing detection of recombinant RoxP by antibodies 2H2F3, 3E10E3, 4D8H1, and 5F3D11 at different antibody concentrations.

FIG. 1B shows competition ELISA curve showing RoxP detection by 2H2F3, 3E10E3, 4D8H1, and 5F3D11 antibodies in the presence of biotinylated 2H2F3

FIG. 1C shows competition ELISA curve showing RoxP detection by 2H2F3, 3E10E3, 4D8H1, and 5F3D11 antibodies in the presence of biotinylated 4D8H1.

FIG. 1D shows linear range of sandwich ELISA for the detection of RoxP using 4D8H1 and biotinylated 2H2F3 as capture and detection antibody, respectively. All samples were run with three technical replicates. Bars represent Standard Deviation.

FIG. 1E shows indirect ELISA curves showing detection of recombinant RoxP by biotinylated antibodies b-2H2F3, b-3E10E3, b-4D8H1, and b-5F3D11 at different antibody concentrations.

FIG. 2A shows recovery of spiked-in RoxP from biofluids and C. acnes media. 50 ng recombinant RoxP was spiked into serially diluted human serum. Samples were analyzed by sandwich ELISA in duplicates. Each plot shows calculated percent recovery. Horizontal red line indicates 100% recovery.

FIG. 2B shows recovery of spiked-in RoxP from biofluids and C. acnes media. 50 ng recombinant RoxP was spiked into serially diluted human synovial fluid. Samples were analyzed by sandwich ELISA in duplicates. Each plot shows calculated percent recovery. Horizontal red line indicates 100% recovery.

FIG. 2C shows recovery of spiked-in RoxP from biofluids and C. acnes media. 50 ng recombinant RoxP was spiked into serially diluted human cerebrospinal fluid. Samples were analyzed by sandwich ELISA in duplicates. Each plot shows calculated percent recovery. Horizontal red line indicates 100% recovery.

FIG. 2D shows recovery of spiked-in RoxP from biofluids and C. acnes media. 50 ng recombinant RoxP was spiked into serially diluted bovine synovial fluid. Samples were analyzed by sandwich ELISA in duplicates. Each plot shows calculated percent recovery. Horizontal red line indicates 100% recovery.

FIG. 2E shows recovery of spiked-in RoxP from biofluids and C. acnes media. 50 ng recombinant RoxP was spiked into serially diluted RCM media. Samples were analyzed by sandwich ELISA in duplicates. Each plot shows calculated percent recovery. Horizontal red line indicates 100% recovery.

FIG. 2F shows recovery of spiked-in RoxP from biofluids and C. acnes media. 50 ng recombinant RoxP was spiked into serially diluted BH1 media. Samples were analyzed by sandwich ELISA in duplicates. Each plot shows calculated percent recovery. Horizontal red line indicates 100% recovery.

FIG. 3A shows a multiple sequence alignment of RoxP orthologues. Residue numbering of the nascent polypeptide chain and predicted, mature, secreted RoxP is shown. RoxP signal peptide cleavage was predicted by SignalP-4.1. All residue positions noted refer to mature, secreted RoxP (i.e., post-signal peptide cleavage) numbering. Residue backgrounds are colored according to sequence identity and variable positions are highlighted in white. The surface-exposure of each residue's side chain in the apo-RoxP NMR structure (PDB 7bcj) as calculated by GetArea3 is shown below the alignment as a percentage exposure compared to the same residue in a tripeptide (Gly-X-Gly) random coil. Variant positions within predicted, mature, secreted RoxP include: 38 total non-conservative variant positions (27%), 31 variant positions with exclusively non-conservative variants (22%), 27 total conservative/semi-conservative variant positions (19%), 20 variant positions with exclusively conservative/semi-conservative variants (14%), and 7 variant positions (23, 33, 53, 56, 103, 142, 160) with both non-conservative and conservative/semi-conservative variants (5%). Solvent-accessibility categories: Buried (≤1%), Partially-buried (>1% to <30%), Partially-accessible (30-60%), Solvent-exposed (>60%). The first digit of a residue position was aligned to the corresponding MSA column e.g., 1 of 10 is centered on the corresponding MSA column for position 10. The sequences depicted in the figure are amino acids 23 to 139 of SEQ NOs: 79-89, respectively.

FIG. 3B shows polyacrylamide gels with enrichment of RoxP at each step in the purification process of RoxP.

FIG. 3C shows the data for ten recombinant RoxP orthologues that were evaluated for binding by 2H2F3, 3E10E3, 4D8H1, and 5F3D11. Absorbance signals were normalized and plotted.

FIG. 3D shows predicted binding regions based on indirect ELISA results for 2H2F3, 3E10E3, 4D8H1, and 5F3D11 antibodies based on RoxP solution structure (PDB: 7BCJ).

FIG. 3E shows RoxP solution structure. All non-conservative variants of RoxP are shown on the the RoxP solution structure (PDB: 7BCJ). Variants are colored according to their surface exposure. Black, surface buried; Grey, partially exposed; White, surface exposed.

FIG. 3F is series of bar graph showing absorbance depicting detecting by antibodies tested. Ten recombinant RoxP orthologs (10 μg/mL) were evaluated for binding by 2H2F3, 3E10E3, 4D8H1, and 5F3D11. Detection was done by poly-HRP Absorbance signals were normalized and plotted.

FIGS. 3G-3H are a series of bar graphs showing absorbance at 450-57 nm depicting detecting by antibodies tested. Ten recombinant RoxP orthologs (1 μg/mL) were evaluated for binding by 2H2F3, 3E10E3, 4D8H1, and 5F3D11. Detection was performed using SA-HRP.

FIG. 3I shows RoxP solution structure (PDB: 7BCJ) with predicted binding regions based on indirect ELISA results for 2H2F3 (blue), 3E10E3 (red), 4D8H1 (green), and 5F3D11 (purple) antibodies.

FIG. 3J shows multiple sequence alignment of 21 RoxP orthologs. Residue backgrounds are colored according to sequence identity. The sequences depicted in the figure have SEQ ID NOs: 79-99, respectively.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The following detailed description references the accompanying drawings that illustrate various embodiments of the present inventive concept. The drawings and description are intended to describe aspects and embodiments of the present inventive concept in sufficient detail to enable those skilled in the art to practice the present inventive concept. Other components can be utilized and changes can be made without departing from the scope of the present inventive concept. The following description is, therefore, not to be taken in a limiting sense. The scope of the present inventive concept is defined only by the appended claims, along with the full scope of equivalents to which such claims are entitled.

The present disclosure is based, in part, on the identification of a panel of mouse monoclonal antibodies that specifically target the RoxP protein of Cutibacteria. RoxP is a 16 kDa secreted protein unique to the Cutibacterium sp. and is important for its antioxidant properties for maintenance of healthy skin. Antibodies as provided herein can be used for efficient detection of Cutibacterium sp. with minimal signal from contaminating bacteria. Accordingly, in some aspects, the current disclosure provides epitope binding agents, for example an antibody or a fragment/variant/conjugate thereof, that specifically binds to RoxP. In some aspects, the current disclosure encompasses compositions, for example diagnostic compositions, and pharmaceutical compositions, comprising these epitope binding agents or antibodies. In some aspects, the current disclosure also provides methods of making these epitope binding agents and compositions thereof. In some aspects, the current disclosure also provides methods of using the compositions provided herein in clinical and non-clinical applications.

### I. Terminology

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991), all of which are incorporated by reference herein. As used herein, the following terms have the meanings ascribed to them below, unless specified otherwise.

The phraseology and terminology employed herein are for the purpose of description and should not be regarded as limiting. When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements. Wherever the terms “comprising” or “including” are used, it should be understood the disclosure also expressly contemplates and encompasses additional aspects “consisting of” the disclosed elements, in which additional elements other than the listed elements are not included.

The term “about” or “approximately,” as used herein, can mean within an acceptable error range for the particular value as determined by one of ordinary skill in the art, which will depend in part on how the value is measured or determined, e.g., the limitations of the measurement system. For example, “about” can mean within 1 or more than 1 standard deviation, per the practice in the given value. Where particular values are described in the application and claims, unless otherwise stated the term “about” can mean an acceptable error range for the particular value, such as 10% of the value modified by the term “about.” As used herein, the term “about,” can mean relative to the recited value, e.g., amount, dose, temperature, time, percentage, etc., ±10%, ±9%, ±8%, ±7%, ±6%, ±5%, ±4%, ±3%, ±2%, or ±1%.

Further, as the present inventive concept is susceptible to aspects of many different forms, it is intended that the present disclosure be considered as an example of the principles of the present inventive concept and not intended to limit the present inventive concept to the specific aspects shown and described. Any one of the features of the present inventive concept may be used separately or in combination with any other feature. References to the terms “aspect,” “aspects,” and/or the like in the description mean that the feature and/or features being referred to are included in, at least, one aspect of the description. Separate references to the terms “aspect,” “aspects,” and/or the like in the description do not necessarily refer to the same aspect and are also not mutually exclusive unless so stated and/or except as will be readily apparent to those skilled in the art from the description. For example, a feature, structure, process, step, action, or the like described in one aspect may also be included in other aspects but is not necessarily included. Thus, the present inventive concept may include a variety of combinations and/or integrations of the aspects described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

The terms “comprising,” “including,” “encompassing” and “having” are used interchangeably in this disclosure. The terms “comprising,” “including,” “encompassing” and “having” mean to include, but not necessarily be limited to the things so described.

The terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

As used herein, the term “treating” refers to the application or administration of a composition including one or more active agents to a subject, who has a target disease or disorder, a symptom of the disease/disorder, or a predisposition toward the disease/disorder, with the purpose to cure, heal, alleviate, relieve, alter, remedy, ameliorate, improve, or affect the disorder, the symptom of the disease, or the predisposition toward the disease or disorder.

Alleviating a target disease/disorder includes delaying the development or progression of the disease or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies using a number of subjects sufficient to give a statistically significant result.

The term “nucleic acid” or “polynucleotide” refers to deoxyribonucleic acids (DNA) or ribonucleic acids (RNA) and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues See, e.g., Batzer et al., Nucleic Acid Res. 19:5081 (1991), the disclosure of which is incorporated in its entirety herein.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

Within the context of the application a protein is represented by an amino acid sequence and correspondingly a nucleic acid molecule or a polynucleotide represented by a nucleic acid sequence. Identity and similarity between sequences: throughout this application, each time one refers to a specific amino acid sequence SEQ ID NO (take SEQ ID NO: Y as example), one may replace it by: a polypeptide represented by an amino acid sequence comprising a sequence that has at least 60%, 70%, 75%, 80%, 85%, 90%, 95, or 99% sequence identity or similarity with amino acid sequence SEQ ID NO: Y.

Each amino acid sequence described herein by virtue of its identity or similarity percentage with a given amino acid sequence respectively has in a further preferred aspect an identity or a similarity of at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% with the given nucleotide or amino acid sequence, respectively. The terms “homology”, “sequence identity” and the like are used interchangeably herein. Sequence identity is described herein as a relationship between two or more amino acid (polypeptide or protein) sequences or two or more nucleic acid (polynucleotide) sequences, as determined by comparing the sequences. In a preferred aspect, sequence identity is calculated based on the full length of two given SEQ ID NO's or on a part thereof. Part thereof preferably means at least 50%, 60%, 70%, 80%, 90%, or 100% of both SEQ ID NO's. In the art, “identity” also refers to the degree of sequence relatedness between amino acid or nucleic acid sequences, as the case may be, as determined by the match between strings of such sequences. The degree of sequence identity between two sequences can be determined, for example, by comparing the two sequences using computer programs commonly employed for this purpose, such as global or local alignment algorithms. Non-limiting examples include BLASTp, BLASTn, Clustal W, MAFFT, Clustal Omega, AlignMe, Praline, GAP, BESTFIT, or another suitable method or algorithm. A Needleman and Wunsch global alignment algorithm can be used to align two sequences over their entire length or part thereof (part thereof may mean at least 50%, 60%, 70%, 80%, 90% of the length of the sequence), maximizing the number of matches and minimizes the number of gaps. Default settings can be used and preferred program is Needle for pairwise alignment (in an aspect, EMBOSS Needle 6.6.0.0, gap open penalty 10, gap extent penalty: 0.5, end gap penalty: false, end gap open penalty: 10, end gap extent penalty: 0.5 is used) and MAFFT for multiple sequence alignment (in an aspect, MAFFT v7Default value is: BLOSUM62 [bl62], Gap Open: 1.53, Gap extension: 0.123, Order: aligned, Tree rebuilding number: 2, Guide tree output: ON [true], Max iterate: 2, Perform FFTS: none is used).

“Similarity” between two amino acid sequences is determined by comparing the amino acid sequence and its conserved amino acid substitutes of one polypeptide to the sequence of a second polypeptide. Similar algorithms used for determination of sequence identity may be used for determination of sequence similarity. Optionally, in determining the degree of amino acid similarity, the skilled person may also take into account so-called conservative amino acid substitutions. As used herein, “conservative” amino acid substitutions refer to the interchangeability of residues having similar side chains.

For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having Sulphur-containing side chains include cysteine and methionine. Preferred conservative amino acids substitution groups are: valine-leucine-isoleucine, phenylalanine-tyrosine, lysine-arginine, alanine-valine, and asparagine-glutamine. Substitutional variants of the amino acid sequence disclosed herein are those in which at least one residue in the disclosed sequences has been removed and a different residue inserted in its place. The amino acid change may be conservative. Conservative substitutions for each of the naturally occurring amino acids may be as follows: Ala to Ser; Arg to Lys; Asn to Gln or His; Asp to Glu; Cys to Ser or Ala; Gln to Asn; Glu to Asp; Gly to Pro; His to Asn or Gin; Ile to Leu or Val; Leu to Ile or Val; Lys to Arg; Gln or Glu; Met to Leu or Ile; Phe to Met, Leu or Tyr; Ser to Thr; Thr to Ser; Trp to Tyr; Tyr to Trp or Phe; and, Val to Ile or Leu.

The term “antibody” herein is used in the broadest sense and encompasses various antibody structures, including but not limited to monoclonal antibodies, polyclonal antibodies, recombinant antibody, single domain antibodies, nanobodies, multispecific antibodies (e.g., bispecific antibodies), and antibody fragments so long as they exhibit the desired antigen-binding activity. The term is further defined and elaborated on in the detailed description.

An “antibody fragment” or “epitope binding fragment” or “antigen binding fragment” refers to a molecule other than an intact antibody that comprises a portion of an intact antibody that binds the antigen to which the intact antibody binds. Examples of antibody fragments include, but are not limited to, Fv, Fab, Fab′, Fab′-SH, F(ab′) 2; diabodies; linear antibodies; single-chain antibody molecules (e.g., scFv); and multispecific antibodies formed from antibody fragments. A part or fragment of the antibody may correspond to at least 1%, at least 2%, at least 3%, at least 4%, at least 5%, at least 10%, at least 20%, at least 30%, at least 40% of the length of the disclosed sequence, such as represented by an amino acid sequence with a specific SEQ ID NO, or at least 50%, or at least 60%, or at least 70%, or at least 80%, or at least 90% of the length.

An “epitope binding agent” comprises an antibody, or epitope binding fragment or a variant and/or conjugate thereof which binds to the secreted protein RoxP. In some aspects, the epitope binding agent can comprise isolated antibody. In some aspects, the epitope binding agent can comprise at least an epitope binding fragment of the antibody disclosed herein. In some aspects, the epitope binding agent comprising at least an epitope binding fragment of one or more of the antibodies disclosed herein may be further conjugated with other molecules as disclosed herein.

An antibody or epitope binding agent “which binds” an antigen of interest, e.g., RoxP protein or a fragment or variant thereof, is one that binds the antigen with sufficient affinity such that the antibody/epitope binding agent is useful as an assay reagent, e.g., as a capture or as a detection antibody/epitope binding agent. Typically, such an antibody/epitope binding agent does not significantly cross-react with other polypeptides. With regard to the binding of a polypeptide to a target molecule, the term “specific binding” or “specifically binds to” or is “specific for” a particular polypeptide or an epitope on a particular polypeptide target means binding that is measurably different from a non-specific interaction. An antibody that “specifically binds” to an antigen or an epitope is a term well understood in the art. A molecule is said to exhibit “specific binding” if for example it reacts more frequently, with more avidity, more rapidly, with greater duration, and/or with greater affinity with a particular target antigen than it does with alternative targets. As an example, an antibody that specifically (or preferentially) binds to an antigen (e.g., RoxP) or an antigenic epitope therein is an antibody that binds this target antigen with greater affinity, avidity, more readily, and/or with greater duration than it binds to other antigens or other epitopes in the same antigen. Specific binding can be measured, for example, by determining binding of a target molecule compared to binding of a control molecule, which generally is a molecule of similar structure that does not have binding activity.

As used herein, “binding affinity” refers to the apparent association constant or K_{A}. The K_{A} is the reciprocal of the dissociation constant (K_{D}).

A “capture antibody,” as used herein, refers to an antibody that specifically binds a target molecule, e.g., a form of RoxP (fragment, derivative or variant thereof), in a sample. Under certain conditions, the capture antibody forms a complex with the target molecule such that the antibody-target molecule complex can be separated from the rest of the sample. In certain aspects, such separation may include washing away substances or material in the sample that did not bind the capture antibody. In certain aspects, a capture antibody may be attached to a solid support surface, such as, for example but not limited to, a plate or a bead, e.g., a paramagnetic bead. In some aspects the capture antibody may be conjugated to a detection molecule or label. Examples of detection labels and conjugates are provided herein throughout the application.

A “detection antibody,” for example a “secondary antibody” as used herein, refers to an antibody that specifically binds a target molecule in a sample or in a sample-capture antibody combination material. Under certain conditions, the detection antibody forms a complex with the target molecule or with a target molecule-capture antibody complex. A detection antibody is capable of being detected either directly through a detection molecule or label or dye, which may be amplified, or indirectly, e.g., through use of another antibody that is labeled and that binds the detection antibody. For direct labeling, the detection antibody is typically conjugated to a moiety that is detectable by some means, for example, including but not limited to, biotin, horse radish peroxidase (HRP), fluorescent molecules, radioactive isotopes etc. Other examples of detection labels and conjugates are provided herein.

The term “chimeric” antibody refers to an antibody in which a portion of the heavy and/or light chain is derived from a particular source or species, while the remainder of the heavy and/or light chain is derived from a different source or species.

The term “monoclonal antibody,” as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies and is not to be construed as requiring production of the antibody by any particular method. For example, the monoclonal antibodies to be used in accordance with the presently disclosed subject matter may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hyper-variable regions (CDRs). See, e.g., Kindt et al. Kuby Immunology, 6th ed., W.H. Freeman and Co., page 91 (2007), the disclosure of which is incorporated in its entirety herein. A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J Immunol. 150:880-887 (1993); Clarkson et al., Nature 352:624-628 (1991), the disclosures of which are incorporated in their entirety herein.

The terms “host cell,” “host cell line,” and “host cell culture” as used interchangeably herein, refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell but may contain mutations. Mutant progeny that has the same function or biological activity as screened or selected for in the originally transformed cell are included herein. In some aspects the host cell may be a hybridoma.

The terms “label” or “detectable label,” or “detection molecule” or “detectible molecule” or “conjugate” as used herein, refers to any chemical group or moiety that can be linked to a substance that is to be detected or quantitated, e.g., an antibody. A label is a detectable label that is suitable for the sensitive detection or quantification of a substance. Non-limiting examples of detectable labels include, but are not limited to, luminescent labels, e.g., fluorescent, phosphorescent, chemiluminescent, bioluminescent and electrochemiluminescent labels, radioactive labels, enzymes, particles, magnetic substances, electroactive species and the like. Alternatively, a detectable label may signal its presence by participating in specific binding reactions. Non-limiting examples of such labels include haptens, antibodies, biotin, streptavidin, his-tag, nitrilotriacetic acid, glutathione S-transferase, glutathione and the like. Further specific examples are provided in the current disclosure.

The term “detection means” as used herein, refers to a moiety or technique used to detect the presence of the detectable antibody through signal reporting that is then read out in an assay. Typically, a detection means employ reagents, e.g., a detection agent, that amplify an immobilized label such as the label captured onto a microtiter plate, e.g., avidin, streptavidin-HRP or streptavidin-β-D-galactopyranose.

The term “detecting,” is used herein, to include both qualitative and quantitative measurements of a target molecule, e.g., RoxP or processed forms thereof. In certain aspects, detecting includes identifying the mere presence of the target molecule in a sample as well as deter-mining whether the target molecule is present in the sample at detectable levels.

The term “sample” is used herein to encompass both biological and non-biological samples. In some aspects the sample may be a clinical sample. In some aspects the sample may be a non-clinical sample. In some aspects the sample may be a laboratory sample. The term sample includes both fluid and non-fluid samples. Examples of fluid samples include but are not limited to biological fluids, cell lysates, non-biological fluids like buffers etc. Non-limiting sources of a biological sample for use in the present invention include cells, solid tissue, biopsy, ascites, aspirates, fluidic extracts, blood (including circulating cells), plasma, serum, spinal fluid, cerebrospinal fluid, lymph fluid, the external sections of the skin, respiratory, intestinal, and genitourinary tracts, tears, saliva, sebum, sweat, milk, tumors, organs, cell cultures and/or cell culture constituents, for example. Methods for obtaining tissue samples and body fluids from animals (e.g., humans) are well known in the art. Non-fluid samples include but are not limited to cells, tissue samples, samples bound to matrices, strips, solid substrate material or membrane (e.g., plastic, nylon, paper), plates etc.

An “individual” or “subject,” as used interchangeably herein, is a mammal. In certain aspects, the individual or subject is a human.

As used herein, the term “populational average” may refer to a population at large; i.e., meaning one or more patients in the local, regional or national population in which the subject resides. The term may also refer to one or more patients in a local, regional or national population in which the subject resides who have undergone surgery, neurosurgery, joint prostheses, neurosurgical shunt or prosthetic heart valves implantation. The term may also refer to one or more healthy patients in a local, regional or national population in which the subject resides.

It should also be understood that, unless clearly indicated to the contrary, in any methods disclosed herein that include more than one step or act, the order of the steps or acts of the method is not necessarily limited to the order in which the steps or acts of the method are recited.

### II. Compositions

In some aspects the current disclosure encompasses compositions comprising isolated antibodies, epitope binding fragment, epitope binding agent, or variant and conjugates thereof and/or nucleic acid sequences encoding the disclosed antibodies, epitope binding fragment, epitope binding agent, or variant and conjugates thereof that specifically bind RoxP.

Antibodies and Epitope binding agents

In some aspects the antibodies or epitope binding agents provided herein comprise at least one amino acid sequence at least about 80% identical to SEQ ID NOS: 1-8, or at least 60% identical to one of SEQ ID NOS: 9-32. In some aspects the antibody or epitope binding agent comprises at least one amino acid sequence at least about 80 to about 85%, or about 85% to about 90%, or about 90% to about 95%, or about 95% to about 100% identical to one of SEQ ID NOS: 1-8 or at least about 60% to about 65%, or about 65% to about 70%, or about 70% to about 75%, or about 75% to about 80% or about 80 to about 85%, or about 85% to about 90%, or about 90% to about 95%, or about 95% to about 100% identical to one or more of SEQ ID NOS: 9-32. In some exemplary aspects the epitope binding agents provided herein recognize certain sequence and/or structural features of RoxP. In some exemplary aspects the epitope binding agents provided herein recognize structural features of Cutibacterial RoxP protein, or a fragment, derivative or variant thereof.

TABLE 1
Amino acid sequences

| SEQ ID | | |
|---|---|---|
| NO | Name | Sequence |
| 1 | V_{H} (5F3D11-1) | MDRLTSSFLLLIVPVYVLSQVTLKESGPGILQPSQTLSLTCSFSGFSL |
| | | STFGMGVSWIRQPSGKGLEWLAHIYWDDDKHYNPSLKSRLTISKDT |
| | | SNNQVFLKITTVDTADTATYYCARRAWGDYDAFAYWGQGTLVTVSA |
| 2 | V_{H} (3E10E3-1) | MGRLTSSFLLLIVPAYVLSQVTLKESGPGILQPSQTLSLTCSFSGFSL |
| | | STYGIGVGWIRQPSGKGLEWLAHIWWNDNKYYNTALKSRLTISKDT |
| | | SNNQVFLKIASVDTADTATYYCARSLPYFDYWGQGTTLTVSS |
| 3 | V_{H} (2H2F3-1) | MGWSCIIFFLVATATGVHSQVQLQQSGAELVRPGVSVKISCKGSGY |
| | | TFTDYAMHWVKQSHAKSLEWIGIISTYYGNASYNQKFKGKATMTVD |
| | | KSSSTAYMELARLTSEDSAIYYCARVPYYGSSYAMDYWGQGTSVTV |
| | | SS |
| 4 | V_{H} (4H8H1-1) | MDFGLIFFIVALLKGVQCEVKLLESGGGLVQPGGSLKLSCAASGFDF |
| | | SRYWMSWVRQAPGKGLEWIGEINPDSSTINYTPSLKDKFIISRDNAK |
| | | NTLYLQMSKVRSEDTALYYCARYYSRFAYWGQGTLVTVSA |
| 5 | V_{L} (5F3D11-1) | EVOLVESGGGLVKPGGSRKLSCAASGFTLSDYGMHWVRQAPDKGL |
| | | EWIAYISSDSNTIHYTDTVKGRFTISRDNARNTLFLQMTSLRSEDTAM |
| | | YYCTRNYGMDYWGQGTS |
| 6 | V_{L} (3E10E3-1) | VLTQSPAIMSASLGERVTMTCTASSSVSSTYLHWYQQNPGSSPKLW |
| | | IYSASNLASGVPTRFSGSGSGTSYSLTISSMEAEDAATYYCHQFHRS |
| | | PYTFGGGTK |
| 7 | V_{L} (2H2F3-1) | MKLPVRLLVLMFWIPASSSDVLMTQTPLSLPVSLGDQASISCRSSQN |
| | | IVHSDGNTYLEWYLQKPGQSPKLLIYKVSNRFSGVPDRFSGSGSGT |
| | | DFTLKISRVEAEDLGVYYCFQGSHVPWTFGGGTKLEIK |
| 8 | V_{L} (4H8H1-1) | MDFGLIFFIVALLKGVQCEVKLLESGGGLVQPGGSLKLSCAASGFDF |
| | | SRYWMSWVRQAPGKGLEWIGEINPDSSTINYTPSLKDKFIISRDNAK |
| | | NTLYLQMSKVRSEDTALYYCARYYSRFAYWGQGTLVTVSA |
| 9 | V_{H} (5F3D11-1)- | TFGMGVS |
| | CDR1 | |
| 10 | V_{H} (5F3D11-1)- | HIYWDDDKHYNPSLKS |
| | CDR2 | |
| 11 | V_{H} (5F3D11-1)- | RAWGDYDAFAY |
| | CDR3 | |
| 12 | V_{L} (5F3D11-1)- | RSSQSLVHSNGNTYLH |
| | CDR1 | |
| 13 | V_{L} (5F3D11-1)- | KVSNRFS |
| | CDR2 | |
| 14 | V_{L} (5F3D11-1)- | SQSTHVPPT |
| | CDR-3 | |
| 15 | V_{H} (3E10E3-1)- | TYGIGVGW |
| | CDR1 | |
| 16 | V_{H} (3E10E3-1)- | HIWWNDNKYYNTALKS |
| | CDR2 | |
| 17 | V_{H} (3E10E3-1)- | SLPYFDY |
| | CDR3 | |
| 18 | V_{L} (3E10E3-1)- | RASENIYSYLA |
| | CDR1 | |
| 19 | V_{L} (3E10E3-1)- | NAKTLAE |
| | CDR2 | |
| 20 | V_{L} (3E10E3-1)- | QHHYGIPRT |
| | CDR-3 | |
| 21 | V_{H} (2H2F3-1)- | DYAMHW |
| | CDR1 | |
| 22 | V_{H} (2H2F3-1)- | IISTYYGNASYNQKFKG |
| | CDR2 | |
| 23 | V_{H} (2H2F3-1)- | VPYYGSSYAMDY |
| | CDR3 | |
| 24 | V_{L} (2H2F3-1)- | DVLMTQTPLSLPVSLGDQASISC |
| | CDR1 | |
| 25 | V_{L} (2H2F3-1)- | KVSNRFS |
| | CDR2 | |
| 26 | V_{L} (2H2F3-1)- | FQGSHVPWT |
| | CDR-3 | |
| 27 | V_{H} (4H8H1-1)- | RYWMS |
| | CDR1 | |
| 28 | V_{H} (4H8H1-1)- | EINPDSSTINYTPSLKD |
| | CDR2 | |
| 29 | V_{H} (4H8H1-1)- | YYSRFAY |
| | CDR3 | |
| 30 | V_{L} (4H8H1-1)- | RSSTGAVTTRNYAN |
| | CDR1 | |
| 31 | V_{L} (4H8H1-1)- | DTNNRAP |
| | CDR2 | |
| 32 | V_{L} (4H8H1-1)- | ALWYSNHWV |
| | CDR-3 | |
| 65 | Sig peptide 1 | MDRLTSSFLLLIVPVYVLS |
| 66 | Sig peptide 2 | MKLPVRLLVLMFWIPASSS |
| 67 | Sig peptide 3 | MGRLTSSFLLLIVPAYVLS |
| 68 | Sig peptide 4 | MSVPTQVLGLLLLWLTGARC |
| 69 | Sig peptide 5 | MGWSCIIFFLVATATGVHS |
| 70 | Sig peptide 6 | MDFGLIFFIVALLKGVQC |
| 71 | Sig peptide 7 | MAWISLILSLLALSSGAIS |

In some exemplary aspects the epitope binding agent is an isolated antibody, or variants, conjugates or fragments thereof that specifically binds RoxP. An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (e.g., full-length comprising the Fc and Fab regions) polyclonal or monoclonal antibodies, but also antigen-binding fragments thereof (such as Fab, Fab′, F(ab′) 2, Fv), single-chain antibody (scFv), fusion proteins comprising an antibody portion (e.g., chimeric antigen receptor or CAR), humanized antibodies, chimeric antibodies, diabodies, single domain antibody, nanobody (e.g., a VH only antibody), multispecific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. Furthermore, the term relates to modified and/or altered antibody molecules, as well as to recombinantly or synthetically generated/synthesized antibodies. The term “antibody” also comprises bifunctional antibodies, trifunctional antibodies, chimeric antibodies or antibody-fusion proteins. An antibody encompassed herein may include an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. In some exemplary aspects, the antibody is an IgG.

In some aspects the term “antibody” as used herein also comprises recombinant antibodies. The term “recombinant antibody” includes all antibodies that are prepared, expressed, created or isolated by recombinant means. Recombinant antibodies are e.g. antibodies obtained by B-cell PCR, or antibodies isolated from an animal (e.g., a mouse) that is transgenic for human immunoglobulin genes, antibodies expressed using a recombinant expression vector transfected into a host cell, antibodies isolated from a recombinant, combinatorial human antibody library, or antibodies prepared, expressed, created or isolated by any other means that involves splicing of human immunoglobulin gene sequences to other DNA sequences. Recombinant rabbit antibodies as produced by B-cell PCR have variable and constant regions (if present) derived from rabbit germline immunoglobulin sequences. i.e., the direct result of B-cell PCR are the binding relevant fragments of an antibody, and the skilled artisan has no problem whatsoever to e.g. construe a full length antibody, a chimeric antibody, or whatever “antibody” that will be desired/required. In some aspects the current disclosure encompasses recombinant antibodies comprising at least one amino acid sequence provided in one of SEQ ID NOS: 1-32.

An antibody molecule encompassed herein may comprise a heavy chain variable region (VH) and a light chain variable region (VL). The VH and VL regions can be further subdivided into regions of hypervariability, also known as “complementarity determining regions” (“CDR”), interspersed with regions that are more conserved, which are known as “framework regions” (“FR”). Each VH and VL may be composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the AbM definition, and/or the contact definition, all of which are well known in the art. See, e.g., Kabat, E. A., et al. (1991) U.S. Department of Health and Human Services, NIH Publication No. 91-3242; Chothia et al., (1989) Nature 342:877; Chothia, C. et al. (1987) J. Mol. Biol. 196:901-917, Al-lazikani et al (1997) J. Molec. Biol. 273:927-948; and Almagro, J. Mol. Recognit. 17:132-143 (2004), the disclosures of which are incorporated in their entirety herein.

In some aspects, the current disclosure encompasses antibodies that comprise at least one amino acid sequence at least about 80% identical to one of the sequences provided in Table 1. In some aspects the isolated antibody provided herein comprises a sequence that is at least 80%, or 81%, or 82%, or 83%, or 84%, or 85%, or 86%, or 87%, or 88%, or 89%, or 90%, or 91%, or 92%, or 93%, or 94%, or 95%, or 96%, or 97%, or 98%, or 99%, or 100% identical to any one or more of SEQ ID NOS: 1-8, or at least 60%, or 61%, or 62%, or 63%, or 64%, or 65%, or 66%, or 67%, or 68%, or 69%, or 70%, or 71%, or 72%, or 73%, or 74%, or 75%, or 76%, or 77%, or 78%, or 79%, or 80%, or 81%, or 82%, or 83%, or 84%, or 85%, or 86%, or 87%, or 88%, or 89%, or 90%, or 91%, or 92%, or 93%, or 94%, or 95%, or 96%, or 97%, or 98%, or 99%, or 100% identical to any one or more of SEQ ID NOS: 9-32. In some aspects the antibody herein may comprise a heavy chain variable region that comprises a sequence that has at least 80% identity to the sequence of any one or more of SEQ ID. NOS: 1-4. In some aspects, the antibody herein may comprise a light chain variable region that comprises a sequence that has at least 80% identity to the sequence of any one of SEQ ID. NOS: 5-8. In some aspects, the antibody herein may comprise a heavy chain CDR that has at least 60% identity to the sequence of any one or more of SEQ ID NOS: 9, 10, 11, 15, 16, 17, 21, 22, 23, 27, 28 or 29. In some aspects the antibody herein may comprise a light chain CDR that has at least 60% identity to the sequence of any one or more of SEQ ID NOS: 12, 13, 14, 18, 19, 20, 24, 25, 26, 30, 31 or 32. In some exemplary aspects, the current disclosure encompasses an antibody comprising a heavy chain variable region having the sequence of SEQ ID NO: 1 and a light chain variable region having the sequence of SEQ ID NO: 5, or a heavy chain variable region having the sequence of SEQ ID NO: 1 and a light chain variable region having the sequence of SEQ ID NO. 6, or a heavy chain variable region having the sequence of SEQ ID NO: 1 and a light chain variable region having the sequence of SEQ ID NO. 7, or a heavy chain variable region having the sequence of SEQ ID NO: 1 and a light chain variable region having the sequence of SEQ ID NO. 8, or a heavy chain variable region having the sequence of SEQ ID NO: 2 and a light chain variable region having the sequence of SEQ ID NO. 5, or a heavy chain variable region having the sequence of SEQ ID NO: 2 and a light chain variable region having the sequence of SEQ ID NO. 6, or a heavy chain variable region having the sequence of SEQ ID NO: 2 and a light chain variable region having the sequence of SEQ ID NO. 7, or a heavy chain variable region having the sequence of SEQ ID NO: 2 and a light chain variable region having the sequence of SEQ ID NO. 8, or a heavy chain variable region having the sequence of SEQ ID NO: 3 and a light chain variable region having the sequence of SEQ ID NO. 5, or a heavy chain variable region having the sequence of SEQ ID NO: 3 and a light chain variable region having the sequence of SEQ ID NO. 6, or a heavy chain variable region having the sequence of SEQ ID NO: 3 and a light chain variable region having the sequence of SEQ ID NO. 7, or a heavy chain variable region having the sequence of SEQ ID NO: 3 and a light chain variable region having the sequence of SEQ ID NO. 8, or a heavy chain variable region having the sequence of SEQ ID NO: 4 and a light chain variable region having the sequence of SEQ ID NO. 5, or a heavy chain variable region having the sequence of SEQ ID NO: 4 and a light chain variable region having the sequence of SEQ ID NO. 6, or a heavy chain variable region having the sequence of SEQ ID NO: 4 and a light chain variable region having the sequence of SEQ ID NO. 7, or a heavy chain variable region having the sequence of SEQ ID NO: 4 and a light chain variable region having the sequence of SEQ ID NO. 8. In some aspects the antibody herein may comprise a heavy chain CDR that has the sequence of SEQ ID NO: 9, 10, 11, 15, 16, 17, 21, 22, 23, 27, 28 or 29. In some aspects the antibody herein may comprise a light chain CDR that has the sequence of any one or more of SEQ ID NOS: 12, 13, 14, 18, 19, 20, 24, 25, 26, 30, 31 or 32.

In an exemplary aspect the antibody provided herein is a monoclonal antibody selected from IgG-5F3D11-1, IgG-3E10E3-1, IgG-2H2F3-1 or IgG-4D8H1-1 or a conjugate or variant thereof that specifically binds one or more structural epitopes on RoxP as disclosed herein in the examples. In certain aspects, epitope binding agents or antibodies described herein may specifically bind to a corresponding target antigen (e.g., RoxP) or an epitope thereof. In some aspects the antibodies provided herein recognize certain sequence and structural features of Cutibacteria RoxP or fragments, variants or derivatives thereof.

In some aspects, the antibodies as disclosed herein may comprise a signal peptide. Some exemplary signal peptides are provided in Table 1, and may have one of SEQ ID NOS: 65-71.

In certain aspects, antibodies described herein may have a suitable binding affinity for a target antigen (e.g. RoxP). In some aspects, an antibody described herein may have a binding affinity (K_{D}) of at least about 1000 nM, at least about 100 nM, at least about 10 nM, at least about 1 nM, at least about 0.1 nM, or lower for an epitope of RoxP or a variant or derivative thereof. In some aspects, an antibody described herein may have a binding affinity (K_{D}) between about 1000 nM to about 0.1 nM (e.g., about 1000 nM, about 750 nM, about 500 nM, about 250 nM, about 100 nM, about 75 nM, about 50 nM, about 25 nM, about 10 nM, about 5 nM, about 1 nM, about 0.75 nM, about 0.5 nM, about 0.25 nM, about 0.1 nM) for RoxP or a variant or derivative thereof. In some aspects, an antibody described herein may have a binding affinity (K_{D}) between about 50 nM to about 40 nM (e.g., about 50 nM, about 40 nM, about 30 nM, about 10 nM, about 1 nM, about 0.8 nM, about 0.6 nM, about 0.4 nM, about 0.3 nM, about 0.2 nM, about 0.1 nM) for a epitope on RoxP or a variant or derivative thereof. In some aspects, binding affinity (or binding specificity) can be determined by a variety of methods including equilibrium dialysis, equilibrium binding, gel filtration, ELISA, surface plasmon resonance, and/or spectroscopy (e.g., using a fluorescence assay).

In some aspects, the heavy chain of the antibody disclosed herein may further comprise a heavy chain constant region (CH) or a portion thereof (e.g., CH1, CH2, CH3, or a combination thereof). In some aspects, a heavy chain constant region for use herein may be of any suitable origin, e.g., human, mouse, rat, or rabbit. In some aspects, alternatively or in addition, a light chain of any of the antibodies disclosed herein may further comprise a light chain constant region (CL), which can be any CL known in the art. In some aspects, a CL may be a kappa light chain. In some aspects, a CL may be a lambda light chain. Antibody heavy and light chain constant regions are well known in the art, e.g., those provided in the IMGT database (www.imgt.org) or at www.vbase2.org/vbstat.php., both of which are incorporated by reference herein.

In some aspects, the antibody may be a full-length antibody or an antigen-binding fragment thereof. In some aspects, isolated antibodies herein may be a full-length antibody, which is an IgG molecule. In some aspects, isolated antibodies herein may be a Fab, a (Fab′) 2, and/or a single-chain antibody. In some aspects, antibodies disclosed herein may be a single chain antibody (scFv). In some aspects, scFv antibody herein may comprise a VH fragment and a VL fragment, which may be linked via a linker. In accordance with these aspects, a linker incorporated between the two variable regions herein may be a flexible linker, a rigid linker, a cleavable linker, or any combination thereof. In some aspects, a linker incorporated between the two variable regions herein may be a flexible peptide linker, a rigid peptide linker, a cleavable peptide linker, or any combination thereof. In accordance with these aspects, a peptide linker incorporated between the two variable regions herein may be at least one amino acid. In some aspects, a peptide linker incorporated between the two variable regions herein may be about 1 amino acid to about 50 amino acids (e.g., about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, about 32, about 34, about 36, about 38, about 40, about 42, about 44, about 46, about 48, about 50). In some aspects, a scFv antibody herein may comprise a VH fragment and a VL fragment, which may be linked via a flexible peptide linker. In some exemplary aspects the antibody herein may comprise a heavy chain variable region having the amino acid sequence of any one of SEQ ID NOS: 1-4 or a light chain variable region having the sequence of any one of SEQ ID NO: 5-9 or both and a linker.

In some aspects, a scFv antibody herein may be in the V_{H}→V_{L} orientation (from N-terminus to C-terminus). In some aspects, a scFv antibody herein may be in the V_{L}→V_{H} orientation (from N-terminus to C-terminus).

In some aspects, antibodies herein can be characterized by identifying an epitope or more than one epitope to which the antigen binds, or “epitope mapping.” There are many methods known in the art for mapping and characterizing the location of epitopes on proteins, including, but not limited to, solving the crystal structure of an antibody-antigen complex, cryo-EM, competition assays, gene fragment expression assays, and synthetic peptide-based assays. In some aspects, epitope mapping can be used to determine the sequence, to which an antibody bind. In some particular aspects of the current disclosure the epitope comprises structural features of RoxP as disclosed in Example 4.

In some aspects the current disclosure also encompasses epitope binding agent or antibodies disclosed herein conjugated to a detector molecule (labels, dyes, assay molecules) for example fluorescent labels, phosphorescent molecules, chemiluminescent molecules, chromophores, luminescent molecules, photoaffinity molecules, colored particles and/or ligands, such as biotin fluorescent dyes, electrochemiluminescense dyes, metal-chelate complexes or labels.

Examples of fluorescent dyes are described by Briggs et al “Synthesis of Functionalized Fluorescent Dyes and Their Coupling to Amines and Amino Acids,” J. Chem. Soc., Perkin-Trans. 1 (1997) 1051-1058, the disclosure of which is incorporated in its entirety herein. Examples include a fluorescent label or a dye. A fluorescent label comprises a fluorophore, which is a fluorescent chemical compound that can re-emit light upon light excitation. Examples of fluorescent label include, but are not limited to, xanthene derivatives (e.g., fluorescein, rhodamine, Oregon green, eosin, and Texas red), cyanine derivatives (e.g., cyanine, indocarbocyanine, oxacarbocyanine, thiacarbocyanine, and merocyanine), squaraine derivatives and ring-substituted squaraines (e.g., Seta and Square dyes), squaraine rotaxane derivatives such as SeTau dyes, naphthalene derivatives (e.g., dansyl and prodan derivatives), coumarin derivatives, oxadiazole derivatives (e.g., pyridyloxazole, nitrobenzoxadiazole and benzoxadiazole), anthracene derivatives (e.g., anthraquinones, including DRAQ5, DRAQ7 and CyTRAK Orange), pyrene derivatives such as cascade blue, oxazine derivatives (e.g., Nile red, Nile blue, cresyl violet, and oxazine 170), acridine derivatives (e.g., proflavin, acridine orange, and acridine yellow), arylmethine derivatives (e.g., auramine, crystal violet, and malachite green), and tetrapyrrole derivatives (e.g., porphin, phthalocyanine, and bilirubin). A dye can be a molecule comprising a chromophore, which is responsible for the color of the dye. In some examples, the detectable label can be fluorescein isothiocyanate (FITC), phycoerythrin (PE), biotin, Allophycocyanin (APC) or Alexa Fluor® 488.

Luminescent dyes or labels can be further subcategorized into chemiluminescent and electro-chemiluminescent dyes. The different classes of chemiluminogenic labels include luminol, acridinium compounds, coelenterazine and analogues, dioxetanes, systems based on peroxyoxalic acid and their derivatives.

The labels of major relevance used as electrochemiluminescent labels are the Ruthenium- and the Iridium-based electrochemiluminescent complexes, respectively.

Electrochemiluminescense (ECL) is very useful in analytical applications as a highly sensitive and selective method. It combines analytical advantages of chemiluminescent analysis (absence of background optical signal) with ease of reaction control by applying electrode potential. In general Ruthenium complexes, especially [Ru (Bpy)₃]²⁺ (which releases a photon at ˜620 nm) regenerating with TPA (Tripropylamine) in liquid phase or liquid-solid interface are used as ECL-labels. Electrochemiluminescent (ECL) assays provide a sensitive and precise measurement of the presence and concentration of an analyte of interest. Such techniques use labels or other reactants that can be induced to luminesce when electrochemically oxidized or reduced in an appropriate chemical environment. Such electrochemiluminescence is triggered by a voltage imposed on a working electrode at a particular time and in a particular manner. The light produced by the label is measured and indicates the presence or quantity of the analyte. Recently also Iridium-based ECL-labels have been described.

In one aspect the directly detectable label/molecule is a chemiluminescent or an electrochemiluminescent label. The light produced by the label is measured and directly or indirectly indicates the presence or quantity of the analyte.

Radioactive labels make use of radioisotopes (radionuclides), such as iodine (¹²⁵I, ¹²¹I, ¹²⁴I, ¹³¹I), carbon (¹⁴C, ¹¹C), sulfur (³⁵S), tritium (³H), indium (¹²¹In), Flourine (¹⁸F), Phosphorus (³²P), Copper (⁶⁴Cu), Gallium (⁶⁸Gn), Yittrium (⁸⁶Y), Zirconium (⁸⁹Zr), Technetium (⁹⁹TC), Indum (¹¹¹In), Xenon (¹³³Xe), Lutetium (¹⁷⁷Lu), or Astatine (²¹¹At).

In some aspects, the label is a metal-chelate complex. Metal-chelate complexes suitable as labels for imaging purposes are well-known in the art

In other aspects, derivatization of immunoglobulins by selectively introducing sulfhydryl groups in the Fc region of an immunoglobulin using reaction conditions that do not alter the antibody combining site are contemplated. Antibody conjugates produced according to this methodology are disclosed to exhibit improved longevity, specificity and sensitivity. Site-specific attachment of effector or reporter molecules, wherein the reporter or effector molecule is conjugated to a carbohydrate residue in the Fc region, has also been disclosed in the literature.

In some aspects the epitope binding agent or antibodies provided herein may also be conjugated to a polynucleotide or a nucleic acid. In some aspects the polynucleotide may be a DNA. In some aspects the polynucleotide may be an RNA. In some aspects the polynucleotide may be a modified DNA or RNA. In some aspects the polynucleotide may comprise a radionuclide.

### Polynucleotides and Host Cells

Polynucleotides, vectors, and host cells can be used to prepare any one of the antibodies disclosed herein (e.g., an antibody that binds RoxP) using recombinant technology, as exemplified herein. In some aspects the current disclosure also encompasses polynucleotide sequences that comprise a nucleic acid sequence encoding a polypeptide corresponding one or more of SEQ ID NOS: 1-8 or at least 80% identical to SEQ ID NOS: 1-8. In some aspects the current disclosure also encompasses polynucleotide sequences that comprise a nucleic acid sequence encoding a polypeptide corresponding one or more of SEQ ID NOS: 9-32 or at least 60% identical to SEQ ID NOS: 9-32. In some aspects the polynucleotide sequence may comprise a nucleic acid sequence that is at least about 60% (e.g., about 60%, or about 65%, or about 70%, or about 75%, about 80%, about 85%, about 90%, about 95%, about 98% or 100%) identical to the sequence of any one or more of SEQ ID NO: 33-64 as provided in Table 2. In some aspects, the polynucleotide sequence encodes a VH and comprises a nucleic acid sequence at least about 80% identical to any one of SEQ ID NOS: 33-36. In some aspects, the polynucleotide sequence encodes a VL and comprises a nucleic acid sequence at least about 80% identical to any one of SEQ ID NOS: 37-40. In some aspects, the polynucleotide sequence comprises a nucleic acid sequence at least about 80% identical to any one of SEQ ID NOS: 41-64.

TABLE 2
Nucleic Acid Sequences

| SEQ ID | | |
|---|---|---|
| NO | Name | Sequence |
| 33 | nV_{H} | TATCCCAGGTTACTCTGAAAGAGTCTGGCCCTGGTATATTGCAGCCCTC |
| | (5F3D11-1) | CCAGACCCTCAGTCTGACCTGTTCTTTCTCTGGGTTTTCACTGAGCACT |
| | | TTTGGTATGGGTGTGAGCTGGATTCGTCAGCCTTCAGGGAAGGGTCTG |
| | | GAGTGGCTGGCACACATTTATTGGGATGATGACAAGCACTATAACCCAT |
| | | CCTTGAAGAGCCGGCTCACAATCTCCAAGGATACCTCCAACAACCAGG |
| | | TATTCCTCAAGATCACCACTGTGGACACTGCAGATACTGCCACATACTA |
| | | CTGTGCTCGAAGAGCTTGGGGTGATTACGACGCGTTTGCTTACTGGGG |
| | | CCAAGGGACTCTGGTCACTGTCTCTGCA |
| 34 | nV_{H} | TGCATATGTCCTCTCCCAGGTTACTCTGAAAGAGTCTGGCCCTGGGATA |
| | (3E10E3-1) | TTGCAGCCCTCCCAGACCCTCAGTCTGACTTGTTCTTTCTCTGGGTTTT |
| | | CACTGAGCACTTATGGTATAGGAGTAGGCTGGATTCGTCAGCCTTCAG |
| | | GGAAGGGTCTGGAGTGGCTGGCACACATTTGGTGGAATGATAATAAGT |
| | | ACTATAACACAGCCCTGAAGAGCCGGCTCACAATCTCCAAGGATACCT |
| | | CCAACAACCAGGTATTCCTCAAGATCGCCAGTGTGGACACTGCAGATA |
| | | CTGCCACATACTACTGTGCTCGCTCATTACCCTACTTTGACTACTGGGG |
| | | CCAAGGCACCACTCTCACAGTCTCCTCA |
| 35 | nV_{H} | CAGGTCCAGCTGCAGCAGTCTGGGGCTGAGCTGGTGAGGCCTGGGGT |
| | (2H2F3-1) | CTCAGTGAAGATTTCCTGCAAGGGTTCTGGCTACACATTCACTGATTAT |
| | | GCTATGCACTGGGTGAAGCAGAGTCATGCAAAGAGTCTAGAGTGGATT |
| | | GGAATTATTAGTACTTACTATGGTAATGCTAGCTACAACCAGAAGTTCAA |
| | | GGGCAAGGCCACAATGACTGTAGACAAATCGTCCAGCACAGCCTATAT |
| | | GGAACTTGCCAGACTGACATCTGAGGATTCTGCCATCTATTACTGTGCA |
| | | AGAGTCCCTTACTACGGTAGTAGCTATGCTATGGACTACTGGGGTCAA |
| | | GGAACCTCAGTCACCGTCTCCTCA |
| 36 | V_{H} | GAGGTGAAGCTTCTCGAGTCTGGAGGTGGCCTGGTGCAGCCTGGAGG |
| | (4H8H1-1) | ATCCCTGAAACTCTCCTGTGCAGCCTCAGGATTCGATTTTAGTAGATAC |
| | | TGGATGAGTTGGGTCCGGCAGGCTCCAGGGAAAGGGCTAGAATGGAT |
| | | TGGAGAAATTAATCCAGATAGCAGTACGATAAACTATACGCCATCTCTA |
| | | AAGGATAAATTCATCATCTCCAGAGACAACGCCAAAAATACGCTGTACC |
| | | TGCAAATGAGCAAAGTGAGATCTGAGGACACAGCCCTTTATTACTGTGC |
| | | AAGATACTATTCCCGTTTTGCTTACTGGGGCCAAGGGACTCTGGTCACT |
| | | GTCTCTGCA |
| 37 | nV_{L} | GATGTTGTGATGACCCAAACTCCACTCTCCCTGCCTGTCAGTCTTGGAG |
| | (5F3D11-1) | ATCAAGCCTCCATCTCTTGCAGATCTAGTCAGAGCCTTGTACACAGTAA |
| | | TGGAAACACCTATTTACATTGGTACCTGCAGAAGCCAGGCCAGTCTCCA |
| | | AAGCTCCTGATCTACAAAGTTTCCAACCGATTTTCTGGGGTCCCAGACA |
| | | GGTTCAGTGGCAGTGGATCAGGGACAGATTTCACACTCAAGATCAGCA |
| | | GAGTGGAGGCTGAGGATCTGGGAGTTTATTTCTGCTCTCAAAGTACAC |
| | | ATGTTCCTCCGACGTTCGGTGGAGGCACCAAGCTGGAAATCAAA |
| 38 | nV_{L} | GACATCCAGATGACTCAGTCTCCAGCCTCCCTATCTGCATCTGTGGGA |
| | (3E10E3-1) | GAAACTGTCACCATCACATGTCGAGCAAGTGAGAATATTTACAGTTATT |
| | | TAGCATGGTATCAGCAGAAACAGGGAAAATCTCCTCAGTTCCTGGTCTA |
| | | TAATGCAAAAACCTTAGCAGAAGGTGTGCCATCAAGGTTCAGTGGCAG |
| | | TGGATCAGGCACACAGTTTTCTCTGAAGATCAACAGCCTGCAGCCTGA |
| | | AGATTTTGGGAGTTATTACTGTCAACATCATTATGGTATTCCTOGGACGT |
| | | TCGGTGGAGGCACCAAGTTGGAAATCAAA |
| 39 | nV_{L} | GATGTTTTGATGACCCAAACTCCACTCTCCCTGCCTGTCAGTCTTGGAG |
| | (2H2F3-1) | ATCAAGCCTCCATCTCTTGCAGATCTAGTCAGAACATTGTACATAGTGA |
| | | TGGAAACACCTATTTAGAATGGTACCTGCAGAAACCAGGCCAGTCTCCA |
| | | AAGCTCCTGATCTACAAAGTTTCCAACCGATTTTCTGGGGTCCCAGACA |
| | | GGTTCAGTGGCAGTGGATCAGGGACAGATTTCACACTCAAGATCAGCA |
| | | GAGTGGAGGCTGAGGATCTGGGAGTTTATTACTGCTTTCAAGGTTCACA |
| | | TGTTCCGTGGACGTTCGGTGGAGGCACCAAGCTGGAAATCAAA |
| 40 | nV_{L} | AGGCTGTTGTGACTCAGGAATCTGCACTCACCACATCACCTGGTGAAA |
| | (4H8H1-1) | CAGTCACACTCACTTGTCGCTCAAGTACTGGGGCTGTTACAACTCGTAA |
| | | CTATGCCAACTGGGTCCAAGAAAAACCAGATCATTTATTCACTGGTCTA |
| | | ATAGGTGATACCAACAACCGAGCTCCAGGTGTTCCTGCCAGATTCTCA |
| | | GGCTCCCTGATTGGAGACAAGGCTGCCCTCACCATCACAGGGGCACA |
| | | GACTGAGGATGAGGCAATATATTTCTGTGCTCTATGGTACAGCAACCAT |
| | | TGGGTGTTCGGTGGAGGAACCAAACTGACTGTCCTA |
| 41 | nV_{H} | ACTTTTGGTATGGGTGTGAGC |
| | (5F3D11-1)- | |
| | CDR1 | |
| 42 | nV_{H} | CACATTTATTGGGATGATGACAAGCACTATAACCCATCCTTGAAGAGC |
| | (5F3D11-1)- | |
| | CDR2 | |
| 43 | nV_{H} | AGAGCTTGGGGTGATTACGACGCGTTTGCTTAC |
| | (5F3D11-1)- | |
| | CDR3 | |
| 44 | nV_{L} | AGATCTAGTCAGAGCCTTGTACACAGTAATGGAAACACCTATTTACAT |
| | (5F3D11-1)- | |
| | CDR1 | |
| 45 | nV_{L} | AAAGTTTCCAACCGATTTTCT |
| | (5F3D11-1)- | |
| | CDR2 | |
| 46 | nV_{L} | TCTCAAAGTACACATGTTCCTCCGACGTTCG |
| | (5F3D11-1)- | |
| | CDR-3 | |
| 47 | nV_{H} | ACTTATGGTATAGGAGTAGGC |
| | (3E10E3-1)- | |
| | CDR1 | |
| 48 | nV_{H} | CACATTTGGTGGAATGATAATAAGTACTATAACACAGCCCTGAAGAGC |
| | (3E10E3-1)- | |
| | CDR2 | |
| 49 | nV_{H} | TCATTACCCTACTTTGACTAC |
| | (3E10E3-1)- | |
| | CDR3 | |
| 50 | nV_{L} | CGAGCAAGTGAGAATATTTACAGTTATTTAGCA |
| | (3E10E3-1)- | |
| | CDR1 | |
| 51 | nV_{L} | AATGCAAAAACCTTAGCAGAA |
| | (3E10E3-1)- | |
| | CDR2 | |
| 52 | nV_{L} | CAACATCATTATGGTATTCCTCGGACG |
| | (3E10E3-1)- | |
| | CDR-3 | |
| 53 | nV_{H} | GATTATGCTATGCAC |
| | (2H2F3-1)- | |
| | CDR1 | |
| 54 | nV_{H} | ATTAGTACTTACTATGGTAATGCTAGCTACAACCAGAAGTTCAAGGGC |
| | (2H2F3-1)- | |
| | CDR2 | |
| 55 | nV_{H} | GTCCCTTACTACGGTAGTAGCTATGCTATGGACTAC |
| | (2H2F3-1)- | |
| | CDR3 | |
| 56 | nV_{L} | AGATCTAGTCAGAACATTGTACATAGTGATGGAA |
| | (2H2F3-1)- | |
| | CDR1 | |
| 57 | nV_{L} | AAAGTTTCCAACCGATTTTCT |
| | (2H2F3-1)- | |
| | CDR2 | |
| 58 | nV_{L} | TTTCAAGGTTCACATGTTCCGTGGACG |
| | (2H2F3-1)- | |
| | CDR-3 | |
| 59 | nV_{H} | AGATACTGGATGAGT |
| | (4H8H1-1)- | |
| | CDR1 | |
| 60 | nV_{H} | GAAATTAATCCAGATAGCAGTACGATAAACTATACGCCATCTCTAAAGG |
| | (4H8H1-1)- | AT |
| | CDR2 | |
| 61 | nV_{H} | TACTATTCCCGTTTTGCTTAC |
| | (4H8H1-1)- | |
| | CDR3 | |
| 62 | nV_{L} | CGCTCAAGTACTGGGGCTGTTACAACTCGTAACTATGCCAAC |
| | (4H8H1-1)- | |
| | CDR1 | |
| 63 | nV_{L} | GATACCAACAACCGAGCTCCA |
| | (4H8H1-1)- | |
| | CDR2 | |
| 64 | nV_{L} | GCTCTATGGTACAGCAACCATTGGGTG |
| | (4H8H1-1)- | |
| | CDR-3 | |
| 72 | Signal | ATGGACAGGCTTACTTCCTCATTCCTACTGCTGATTGTCCCTGTCTATG |
| | sequence 1 | TCC |
| 73 | Signal | ATGAAGTTGCCTGTTAGGCTGTTGGTGCTGATGTTCTGGATTCCTGCTT |
| | sequence 2 | CCAGCAGT |
| 74 | Signal | ATGGGCAGGCTTACTTCTTCATTCCTGCTACTGATTGTTCC |
| | sequence 3 | |
| 75 | Signal | ATGAGTGTGCCCACTCAGGTCCTGGGGTTGCTGCTGCTGTGGCTTACA |
| | sequence 4 | GGTGCCAGATGT |
| 76 | Signal | ATGGGTTGGAGCTGTATCATCTTCTTTCTGGTAGCAACAGCTACAGGTG |
| | sequence 5 | TGCACTCC |
| 77 | Signal | ATGGATTTTGGGCTGATTTTTTTTATTGTTGCTCTTTTAAAAGGGGTCCA |
| | sequence 6 | GTGT |
| 78 | Signal | ATGGCCTGGATTTCACTTATACTCTCTCTCCTGGCTCTCAGCTCAGGGG |
| | sequence 7 | CCATTTCC |

In certain aspects, nucleic acids (i.e., polynucleotides) encoding the heavy and light chain of an antibody as described herein can be cloned into one expression vector, each nucleotide sequence being in operable linkage to a suitable promoter. In some aspects, each of the polynucleotide sequences encoding the heavy chain and light chain may be in operable linkage to a distinct promoter. In some aspects, polynucleotide sequences encoding the heavy chain and the light chain may be in operable linkage with a single promoter, such that both heavy and light chains are expressed from the same promoter. In some aspects, when necessary, an internal ribosomal entry site (IRES) can be inserted between the heavy chain and light chain encoding sequences.

In certain aspects, the polynucleotide sequences encoding the heavy chain or light chain or both of the antibodies described herein may have the same polynucleotide sequences encoding the heavy chain or light chain or both as any of the exemplary antibodies described herein (e.g., 5F3D11-1, 3E10E3-1, 2H2F3-1 or 4D8H1-1) or in certain aspects, antibodies disclosed herein may share one or more polynucleotide sequences provided in Table 2. In some exemplary aspects, the polynucleotide may also comprise a signal sequence encoding a signal peptide at the N-terminal of the antibody. In some exemplary aspects, the signal sequence can be any one of SEQ ID NO: 72-78.

In some aspects, genetically engineered antibodies such as single-chain antibodies can be produced via, e.g., conventional recombinant technology or any methods known in the art. In some aspects, polynucleotide encoding a monoclonal antibody specific to a target antigen (e.g. RoxP) can be readily isolated and sequenced using conventional procedures (e.g., by using oligonucleotide probes that are capable of binding specifically to genes encoding the heavy and light chains of the monoclonal antibodies). Once isolated, the polynucleotide sequence can be placed into one or more expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. In some aspects the polynucleotide can be modified, for example, by substituting the coding sequence for human heavy and light chain constant domains in place of the homologous murine sequences, or by covalently joining to the immunoglobulin coding sequence all or part of the coding sequence for a non-immunoglobulin polypeptide. In some aspects, genetically engineered antibodies, such as chimeric or hybrid antibodies; can be prepared that have the binding specificity of a target antigen. In some particular aspects, the current disclosure also encompasses hybridomas producing the epitope binding agents or antibodies described herein. In some exemplary aspects the current disclosure encompasses a hybridoma producing the 5F3D11-1, 3E10E3-1, 2H2F3-1 or 4D8H1-1 monoclonal antibody that specifically binds to epitopes on the extracellular domain of RoxP. In some aspects the current disclosure encompasses a host cell comprising a polypeptide at least 80% identical to any one or more of SEQ ID NOS: 1-32. In some aspects the current disclosure encompasses a host cell comprising a nucleic acid sequence that is at least about 60% (e.g., about 60%, about 65%, about 70%, about 75%, about 80%, about 85%, about 90%, about 95%, about 98%, about 100%) identical to the sequence of any one or more of SEQ ID NOS: 32-64.

In some aspects, a single-chain antibody herein can be prepared via recombinant technology by linking a polynucleotide sequence coding for a heavy chain variable region and a nucleotide sequence coding for a light chain variable region. In some aspects, a linker may be incorporated between the two variable regions. In some aspects, techniques described to produce single chain antibodies can be adapted to produce a phage or yeast scFv library and scFv clones specific to RoxP.

In some aspects, one or more vectors (e.g., expression vectors) having nucleic acids encoding any of the epitope binding agents or antibodies or fragments and variants described herein can be introduced into suitable host cells for producing the antibodies (for example a vector comprising a nucleic acid sequence encoding a polypeptide at least 80% similar to any one or more of SEQ ID NOS: 1-32). In some aspects the current disclosure encompasses vectors comprising a nucleic acid sequence that is at least about 60% (e.g., about 60%, about 65%, about 70%, about 75%, about 80%, about 85%, about 90%, about 95%, about 98%, about 100%) identical to the sequence of any one or more of SEQ ID NOS: 33-64. In some aspects, host cells can be cultured under suitable conditions for expression of the epitope binding agent, antibody or any polypeptide chain or variants thereof. In some aspects, epitope binding agents, antibodies or polypeptide chains, fragments or variants thereof can be recovered from the cultured cells (e.g., from the cells or the culture supernatant) via a conventional method, e.g., affinity purification. In some aspects, polypeptide chains of the antibody herein can be incubated under suitable conditions for a suitable period of time allowing for production of the antibody or functional epitope binding agents.

In certain aspects, standard molecular biology techniques can be used to prepare the recombinant expression vector, transfect the host cells, select for transformants, culture the host cells, and recover the epitope binding agent or antibodies from the culture medium. In some aspects, some antibodies can be isolated by affinity chromatography with a Protein A or Protein G coupled matrix.

### Pharmaceutical and Diagnostic Compositions

In certain aspects, provided herein are pharmaceutical compositions comprising one or more of the antibodies or epitope binding agents disclosed herein. In some aspects, pharmaceutical compositions herein may comprise one or more of the antibodies disclosed herein and at least one pharmaceutically acceptable carrier and/or excipient. Pharmaceutically acceptable carriers or excipients suitable for the compositions described herein are well known to one of skill in the art of use for preserving and delivering antibodies, antibody fragments, epitope binding agent to any mammalian subject including humans and other mammals.

In certain aspects, the pharmaceutical compositions can further include a non-specific innate immune response stimulator mixture or composition or other immunomodulatory agent for enhancing an immune response. In accordance with these aspects, the non-specific innate immune response stimulator can elicit both a cell-mediated immune response and a humoral immune response.

In certain aspects, the pharmaceutical compositions to be used in the present methods can include pharmaceutically acceptable carriers, excipients, and/or stabilizers in the form of lyophilized formulations or aqueous solutions. In some aspects, acceptable carriers, excipients, and/or stabilizers are nontoxic to recipients at the dosages and concentrations used, and can include buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN, PLURONICS or polyethylene glycol (PEG).

In some aspects, the pharmaceutical composition described herein can have liposomes containing the antibodies (or the encoding nucleic acids). In some aspects, liposomes for use herein can be generated by the reverse phase evaporation method with a lipid composition having phosphatidylcholine, cholesterol and PEG-derivatized phosphatidylethanolamine (PEG-PE). In some aspects, liposomes for use herein can be extruded through filters of defined pore size to yield liposomes with the desired diameter.

In some aspects, antibodies, or the encoding nucleic acid(s) herein, can be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions.

In some aspects, the pharmaceutical compositions described herein can be formulated in sustained-release format. In some aspects, pharmaceutical compositions herein to be used for in vivo administration may be sterile. In some aspects, this can be readily accomplished by, for example, filtration through sterile filtration membranes. In some aspects, therapeutic antibody compositions herein can be placed into a container having a sterile access port, for example, an intravenous solution bag or vial having a stopper pierceable by a hypodermic injection needle.

In certain aspects, pharmaceutical compositions described herein can be in unit dosage forms such as tablets, pills, capsules, powders, granules, solutions or suspensions, or suppositories, for oral, parenteral, or rectal administration, or administration by inhalation or insufflation.

In some aspects, emulsion compositions herein can be those prepared by mixing an antibody with Intralipid™ or the components thereof (soybean oil, egg phospholipids, glycerol and water).

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to about 20% oil, for example, between about 5% and about 20%. The fat emulsion can comprise fat droplets between about 0.1 μm and about 1.0 μm, particularly about 0.1 μm and 0.5 μm, and have a pH in the range of about 5.5 to about 8.0.

In some aspects, pharmaceutical compositions herein for inhalation or intranasal administration may include solutions and suspensions in pharmaceutically acceptable, aqueous or organic solvents, or mixtures thereof, and powders. In some aspects, liquid or solid compositions herein can contain suitable pharmaceutically acceptable excipients as set out above. In other aspects, the compositions can be administered by the oral or nasal respiratory route for local or systemic effect.

In some aspects, compositions can be in sterile pharmaceutically acceptable solvents can be nebulized by use of gases. In some aspects, nebulized solutions herein can be breathed directly from the nebulizing device, or the nebulizing device can be attached to a face mask, tent or intermittent positive pressure breathing machine. In some aspects, solution, suspension or powder compositions herein can be administered, preferably orally or nasally, from devices which deliver the formulation in an appropriate manner.

In some aspects, concentrations of antibodies disclosed herein can be a pre-determined concentration or a standard concentration. In some aspects, the antibodies disclosed herein can be in a concentration of about 1 milligrams/milliliters (mg/ml) to about 500 mg/ml, about 1 mg/ml to about 250 mg/ml, about 1 mg/ml to about 200 mg/ml, about 1 mg/ml to about 150 mg/ml, about 1 mg/ml to about 100 mg/ml, about 1 mg/ml to about 75 mg/ml, about 1 mg/ml to about 50 mg/ml, about 0.1 mg/ml to about 100 mg/ml, or other suitable concentration. In some aspects, the antibody is formulated to a concentration of about 30 mg/ml. In some aspects, the antibody is lyophilized. In some aspects, the antibody is diluted in a suitable solution to a suitable concentration prior to administration (e.g., in a therapeutic application described below).

In some aspects the current disclosure also encompasses compositions that comprise one or more of these epitope binding agents or antibodies for in vivo diagnostic use. In some aspects these diagnostic compositions are used in conjunction with in vivo imaging techniques. In some exemplary aspects, epitope binding agents or antibodies may be coupled to dyes, fluorophores or radiolabels as provided above, but for in vivo imaging of RoxP or RoxP interacting proteins. Epitope binding agents of the invention may be administered neat to detect levels of RoxP in vivo in accordance with the present disclosure. More commonly, however, they are administered in the context of acceptable compositions, that contain effective amounts of one or more antibodies together with one or more other ingredients known to those skilled in the art for formulating compositions for in vivo use.

Additional ingredients useful in preparing these in vivo diagnostics in accordance with the present disclosure include, for example, carriers (e.g., in liquid or solid form), flavoring agents, lubricants, solubilizers, suspending agents, fillers, glidants, compression aids, binders, tablet-disintegrating agents, encapsulating materials, emulsifiers, buffers, preservatives, sweeteners, thickening agents, coloring agents, viscosity regulators, stabilizers or osmo-regulators, or combinations thereof.

Liquid diagnostic compositions preferably contain one or more monoclonal antibodies of the invention and one or more liquid carriers to form solutions, suspensions, emulsions, syrups, or pressurized compositions. An acceptable liquid carriers include, for example water, organic solvents, acceptable oils or fat, or combinations thereof. The liquid carrier can contain other suitable additives such as solubilizers, emulsifiers, buffers, preservatives, sweeteners, flavoring agents, suspending agents, thickening agents, colors, viscosity regulators, stabilizers or osmo-regulators, or combinations thereof. If the liquid formulation is intended for pediatric use, it is generally desirable to avoid inclusion of alcohol.

Examples of liquid carriers suitable for oral or parenteral administration include water (preferably containing additives such as cellulose derivatives such as sodium carboxymethyl cellulose), alcohols or their derivatives (including monohydric alcohols or polyhydric alcohols such as glycols) or oils (e.g., fractionated coconut oil and arachis oil). For parenteral administration the carrier can also be an oily ester such as ethyl oleate and isopropyl myristate. The liquid carrier for pressurized compositions can be halogenated hydrocarbons or other acceptable propellant.

### Methods of Using

In some aspects, the current disclosure also encompasses use of the disclosed antibodies for non-clinical or clinical diagnostic purposes. The ability of the antibody provided herein to recognize and bind RoxP, may be utilized in methods for detection of Cutebacteria in in vitro or ex vivo samples and in vivo with suitable administration means.

Assaying for the expression of RoxP protein is intended to include detection, qualitative or quantitative measurement or estimation of the level of RoxP protein or variants or fragments thereof in a sample either directly (e.g., by determining or estimating absolute protein level) or relatively (e.g., by comparing to RoxP protein level in a second sample or standard). RoxP polypeptide expression level in the sample can be measured or estimated and compared to a standard RoxP protein level, the standard being determined from a second biological sample healthy individual or being determined by averaging levels from a population of samples that are not diseased. As will be appreciated in the art, once the “standard” RoxP polypeptide level is known, it can be used repeatedly as a standard for comparison. A standard curve may be created by plotting the mean absorbance against known protein concentration. The standard curve may then be used to determine the concentration of RoxP in a sample.

Samples as used herein may vary depending on the application. For example, it may be a biological sample or a non-biological sample. The term non-biological sample may include synthetic peptides, buffers, non-clinical fluids, artificial antigen bound surfaces etc. The term “biological sample” includes any biological specimen obtained from an individual. Suitable samples for use in the present invention include, without limitation, tissue samples, biopsy, cells, sections, whole blood, plasma, serum, saliva, sweat, sebum, urine, stool (i.e., feces), tears, and any other bodily fluid. One skilled in the art will appreciate that samples can be diluted prior to the analysis of marker levels. The sample may be a fluid sample, a solid sample or a sample bound to a solid surface like matrices, beads, strips, solid substrate material or membrane (e.g., plastic, nylon, paper), plates etc.

The clinical and non-clinical methods of use provided herein share some common principles for detection, qualitative or quantitative measurement. In general, the epitope binding agent or antibody provided herein is contacted with a sample comprising RoxP to form a complex that is either directly detectible due to the presence of a detectible molecule or can be indirectly detected by a detection antibody. Methods to conduct these assays are well established in the art.

In some aspects the current disclosure encompasses methods of using the epitope binding agent and antibodies provided herein to detect and/or assay RoxP protein (for clinical and non-clinical purposes) in a sample using methods known to those of skill in the art, including immunoassays, such as immunohistochemistry (IHC), enzyme linked immunosorbent assay (ELISA), fluorescence-activated cell sorting (FACS), immunohistochemistry (IHC), immunoprecipitation, radioimmunoassays, electrochemiluminescence-based detection assays, magnetic immunoassays, lateral flow assays, and related techniques and Western blotting. Additional suitable immunoassays for detecting the target antigen in a sample will be apparent to those of skill in the art. Methods for performing these assays are known in the art.

In some aspects, anti-RoxP antibodies or antigen-binding fragments thereof described herein can carry a detectable molecule. When radioactive labels are used, currently available counting procedures known in the art may be utilized to identify and quantitate the specific binding of anti-RoxP antibody or antigen-binding protein or fragments thereof to RoxP (e.g. Cutibacterium acnes RoxP). In the instance where the label is an enzyme, detection may be accomplished by any of the presently utilized colorimetric, spectrophotometric, fluorospectrophotometric, amperometric or gasometric techniques as known in the art. This can be achieved by contacting a sample or a control sample with an anti-RoxP antibody or antigen-binding protein of fragments thereof under conditions that allow for the formation of a complex between the antibody or antigen-binding protein or fragment thereof and RoxP. Any complexes formed between the antibody or antigen-binding protein or fragment thereof and RoxP may be detected and compared in the sample and the control. In light of the specific binding of the antibodies or antigen-binding fragments thereof described herein for RoxP, the antibodies or antigen-binding fragments thereof can be used to specifically detect for example RoxP expression. The antibodies or antigen-binding fragments thereof described herein can also be used to purify RoxP via immunoaffinity purification.

The steps of various useful immunodetection methods have been described in the scientific literature. In general, the immunobinding methods include obtaining a sample, e.g. a sample suspected of comprising RoxP, and contacting the sample with a first anti-RoxP antibody in accordance with the present invention under conditions effective to allow the formation of immunocomplexes.

Contacting the chosen biological sample with the antibody under effective conditions and for a period of time sufficient to allow the formation of immune complexes (primary immune complexes) generally comprises adding the antibody composition to the sample and incubating the mixture for a period of time sufficient for the antibodies to form immune complexes with, i.e., to specifically bind to, any RoxP present. After this time, the sample-antibody composition, such as a tissue section, ELISA plate, dot blot or western blot, will generally be washed to remove any non-specifically bound antibody species, allowing only those antibodies specifically bound within the primary immune complexes to be detected

In general, the detection of immunocomplex formation is well known in the art and may be achieved through the application of numerous approaches. These methods are generally based upon the detection of a label or marker, such as for example any of radioactive, fluorescent, biological and enzymatic tags. In some aspects, a secondary binding agent, such as a second antibody and/or a biotin/avidin ligand binding arrangement, may be used in accordance with methodologies known in the art.

In some aspects, the first antibody that becomes bound within the primary immune complexes may be detected by means of a second binding agent that has binding affinity for the antibody. In these cases, the second binding agent may be linked to a detectable label or detectable molecules provided herein. In some aspects, the second binding agent is an antibody, which may thus be termed a “secondary” antibody. The primary immune complexes for example RoxP-epitope binding agent complex are contacted with the labeled, secondary binding agent, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding agent, such as an antibody, that has binding affinity for the antibody is used to form secondary immune complexes, as described above. After washing, the secondary immune complexes are contacted with a third binding agent or antibody that has binding affinity for the second antibody, again under effective conditions and for a period of time sufficient to allow the formation of immune complexes (tertiary immune complexes). The third ligand or antibody is linked to a detectable molecule, allowing detection of the tertiary immune complexes thus formed. This system may provide for signal amplification if this is desired. Thus, in some aspects, any of the primary (epitope binding agent of the current disclosure), secondary or tertiary antibodies may be conjugated to a detectible molecule (examples of which are provided herein).

In some aspects, a biotinylated antibody or epitope binding agent is used to detect the target antigen(s), and a second step antibody is then used to detect the biotin attached to the complexed antibody. In that method the sample to be tested is first incubated in a solution comprising the first step antibody. If the target antigen is present, some of the antibody specifically binds to the antigen to form a biotinylated antibody/antigen complex. The anti-body/antigen complex is then amplified by incubation in successive solutions of streptavidin (or avidin) and biotinylated DNA, and/or complementary biotinylated DNA, with each step adding additional biotin sites to the antibody/antigen complex. The amplification steps are repeated until a suitable level of amplification is achieved, at which point the sample is incubated in a solution comprising the second step antibody against biotin. This second step antibody is labeled, as for example with an enzyme that can be used to detect the presence of the antibody/antigen complex by histoenzymology using a chromogen substrate. With suitable amplification, a conjugate can be produced that is macroscopically visible.

In one aspect, immunohistochemistry (IHC) is used for immunological detection. Using IHC, detection of RoxP in a sample can be achieved by targeting a sample with a binding agent, e.g., an anti-RoxP antibody or antigen-binding fragment thereof. The binding agent can be linked, either directly or indirectly to a detectable label or can be detected by another binding agent that is linked, either directly or indirectly to a detectable label. In one aspect, 3,3′-diaminobenzidine (DAB) is used in the HIC assay to detect the primary antibody bound to RoxP. In one aspect, the concentration of the anti-RoxP antibody or antigen-binding fragment thereof in the IHC assay is about 1 μg/ml to about 50 μg/ml. In one aspect, the concentration of the anti-RoxP antibody or antigen-binding fragment thereof in the IHC assay is about 1 μg/ml to about 20 μg/ml. In one aspect, the concentration of the anti-RoxP antibody or antigen-binding fragment thereof in the IHC assay is about 10 μg/ml.

IHC can be performed on cells, cell pellets, tissues, preparations from blood, plasma, serum, or lymph fluid, etc. In some aspects, the samples are fixed samples. In some aspects, the samples are paraffin embedded samples. In some aspects, the samples are formalin fixed and paraffin embedded samples.

In one aspect, flow cytometry is used for immunological detection. Thus, for example, the number of antibodies bound per cell (ABC) can be assessed using flow cytometry.

In some aspects, the current disclosure also encompasses methods of using the compositions provided herein for in vivo diagnostics applications. In some aspects the compositions provided herein are used in medical imaging of a subject, in need thereof. In some exemplary aspects the method encompasses administering to a subject an imaging effective amount of an in vivo diagnostic composition, wherein the composition comprises the epitope binding agent coupled to a detectable moiety for example a dye or a radiolabel. The localization and accumulation of the imaging agent is then detected. The localization and accumulation of the imaging agent may be detected by for example radionuclide imaging, radio scintigraphy, nuclear magnetic resonance imaging, computed tomography, positron emission tomography, computerized axial tomography, X-ray or magnetic resonance imaging method.

In some aspects, the current disclosure also encompasses a method of treatment in a subject in need thereof, the method comprising administering a therapeutically effective amount of a composition as disclosed herein. In some aspects, the subject is suffering from or likely to suffer from a Cutibacterium infection resulting from a surgery, neurosurgery, joint prostheses, neurosurgical shunt or prosthetic heart valves implantation. In some aspects, the composition can be administered with a pharmaceutically acceptable excipient.

In some aspects, the current disclosure further encompasses a method of treating a subject in need thereof to mitigate or prevent Cutibacterium infection. The method may comprise administering a treatment to the subject, wherein the treatment is suitable for treating the Cutibacterium infection. Also provided are the treatment for use in mitigating or preventing Cutibacterium infection and the treatment in the manufacture of a medicament for mitigating or preventing Cutibacterium infection.

The subject may have been determined to have an amount of Cutibacterium sp above a threshold level. The method may comprise determining an amount of Cutibacterium sp in a sample from the subject and administering to the subject the treatment based on the amount of Cutibacterium sp in the sample. The amount may be above a population average. The subject may be suffering from or likely to suffer from a Cutibacterium infection resulting from a surgery, neurosurgery, joint prostheses, neurosurgical shunt or prosthetic heart valves implantation. The amount of Cutibacterium sp in the sample may be determined using methods disclosed herein. The treatment may comprise a therapeutically effective amount of a composition comprising the isolated antibody, epitope binding fragment, or variant thereof that binds to Rox-P, disclosed herein. In another aspect, the treatment may comprise an antimicrobial agent, a disinfectant, a bacteriocide, an antiseptic, or an astringent.

Non-limiting examples of antimicrobial agent may include vancomycin, cefazolin, cepahalothin, cephalexin, linezolid, daptomycin, clindamycin, lincomycin, mupirocin, bacitracin, neomycin, polymyxin B, gentamicin, prulifloxacin, ulifloxacin, fidaxomicin, minocyclin, metronidazole, metronidazole, sulfamethoxazole, ampicillin, trimethoprim, ofloxacin, norfloxacin, tinidazole, norfloxacin, ornidazole, levofloxacin, nalidixic acid, ceftriaxone, azithromycin, cefixime, ceftriaxone, cefalexin, ceftriaxone, rifaximin, ciprofloxacin, norfloxacin, ofloxacin, levofloxacin, gatifloxacin, gemifloxacin, prufloxacin, ulifloxacin, moxifloxacin, nystatin, amphotericin B, flucytosine, ketoconazole, posaconazole, clotrimazole, voriconazole, griseofulvin, miconazole nitrate, and fluconazole. Disinfectants, bacteriocides, antiseptics, and astringents may include, alcohols (ethyl alcohol, isopropyl alcohol), aldehydes (glutaraldehyde, formaldehyde, formaldehyde-releasing agents (noxythiolin-oxymethylenethiourea, tauroline, hexamine, dantoin), o-phthalaldehyde), anilides (triclocarban=TCC=3,4,4′-triclorocarbanilide), biguanides (chlorhexidine, alexidine, polymeric biguanides (polyhexamethylene biguanides with MW>3,000 g/mol, vantocil), diamidines (propamidine, propamidine isethionate, propamidine dihydrochloride, dibromopropamidine, dibromopropamidine isethionate), phenols (fentichlor, p-chloro-m-xylenol, chloroxylenol, hexachlorophene), bis-phenols (triclosan, hexachlorophene), chloroxylenol (PCMX), quaternary ammonium compounds (cetrimide, benzalkonium chloride, cetyl pyridinium chloride), silver compounds (silver sulfadiazine, silver nitrate), peroxy compounds (hydrogen peroxide, peracetic acid, benzoyl peroxide), iodine compounds (povidone-iodine, poloxamer-iodine, iodine), chlorine-releasing agents (sodium hypochlorite, hypochlorous acid, chlorine dioxide, sodium dichloroisocyanurate, chloramine-T), copper compounds (copper oxide), isotretinoin, sulfur compounds, botanical extracts (Melaleuca spp. (tea tree oil), (Vaccinium spp. (e.g., A-type proanthocyanidins), Cassia fistula Linn, Baekea frutesdens L., Melia azedarach L., Muntingia calabura, Vitis vinifera L, Terminalia avicennioides Guill & Perr., Phylantus discoideus muel. Muel-Arg., Ocimum gratissimum Linn., Acalypha wilkesiana Muell-Arg., Hypericum pruinatum Boiss. & Bal., Hypericum olimpicum L. and Hypericum sabrum L., Hamamelis virginiana (witch hazel), Clove oil, Eucalyptus spp., rosemarinus officinalis spp. (rosemary), thymus spp. (thyme), Lippia spp. (oregano), lemongrass spp., cinnamomum spp., geranium spp., lavendula spp.), aminolevulonic acid, and topical antibiotic compounds (bacteriocins; mupirocin, bacitracin, neomycin, polymyxin B, gentamicin). In certain aspects, the method may further comprise repeating the step of determining an amount of Cutibacterium sp in a sample from the subject at a later time point and/or method of administering to the subject the treatment disclosed herein.

### VI. Kits

In some aspects, the detection methods and compositions of this invention can be provided in the form of a kit. In some aspects, such a kit comprises the antibody or an epitope binding agent or fragment thereof provided herein.

In an exemplary aspect the kit comprises the epitope binding agent disclosed herein; a reporter molecule that detects the RoxP-antibody (epitope binding agent) complex, suitable reagents and instructions for use.

In some exemplary aspects the current disclosure also encompasses diagnostic kits comprising means for obtaining a sample from a subject in need thereof; the epitope binding agent provided herein, a reporter molecule that detects RoxP-antibody (epitope binding agent) complex, reagents and instructions for use.

In some aspects, such a kit is a packaged combination including the basic elements of: a capture antibody comprised of an anti-RoxP antibody; a detectable (labeled or unlabeled) antibody that binds to the antibody of interest and instructions on how to perform the assay method using these reagents.

The kit may further comprise a solid support for the capture reagents, which may be provided as a separate element or on which the capture reagents are already immobilized. In some aspect the capture antibody may already be immobilized on the solid support for example a plate, matrix, paper strip, plastic strip, beads.

Hence, the capture antibodies in the kit may be immobilized on a solid support, or they may be immobilized on such support that is included with the kit or provided separately from the kit. In some aspects, the capture reagents are coated on or attached to a solid material (for example, a microtiter plate, beads or a comb). The detectable antibodies may be labeled antibodies detected directly or unlabeled antibodies that are detected by labeled antibodies directed against the unlabeled antibodies raised in a different species or targeted to another epitope. Where the label is an enzyme, the kit will ordinarily include substrates and cofactors required by the enzyme; where the label is a fluorophore, a dye precursor that provides the detectable chromophore; and where the label is biotin, an avidin such as avidin, streptavidin, or streptavidin conjugated to HRP or β-galactosidase with MUG.

The components of the kit will be provided in predetermined ratios, with the relative amounts of the various reagents suitably varied to provide for concentrations in solution of the reagents that substantially maximize the sensitivity of the assay. Particularly, the reagents may be provided as dry powders, usually lyophilized, including excipients, which on dissolution will provide for a reagent solution having the appropriate concentration for combining with the sample to be tested.

The present disclosure also provides kits for use in treating or alleviating a target disease, such as cancer as described herein. Such kits can include one or more containers comprising an antibody, e.g., any of those described herein. In some aspects, an antibody herein may be co-used with a second therapeutic agent. In some aspects, the kits may further comprise suitable administration means like syringes, intravenous drip apparatus etc.

In some aspects the kits disclosed comprises a container and a label or package insert on or associated with the container. Suitable containers include, for example, bottles, vials, syringes, assay plates, strips, matrices etc. The containers may be formed from a variety of materials such as glass, plastic, paper etc. The kit may further include other materials desirable from a commercial and user standpoint, including other buffers, diluents, filters, needles, and syringes.

A “package insert” is used to refer to instructions customarily included in commercial packages of diagnostic products, that contain information about usage etc.

Instructions included in the kits may be affixed to packaging material or may be included as a package insert. While the instructions are typically written or printed materials, they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” may include the address of an internet site that provides the instructions.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

Cutibacterium acnes, a lipophilic Gram-positive anaerobic bacterium, is one of the major colonizer of the human skin. Even though C. acnes is vital for the regulation of skin homeostasis, it is considered an opportunistic pathogen because it can contribute to the pathogenesis of acne vulgaris and can be isolated from various implant-associated post-operative infections, with periprosthetic joint infections being the most prominent among them. Pre-operative procedures like skin sterilization and prophylactic antibiotics do not effectively reduce C. acnes burden. C. acnes infections of surgical implants are difficult to treat because of the microbe's ability to form biofilms that reduce sensitivity to antimicrobials. In some aspects, the current disclosure encompasses identification of a biomarker for C. acnes infections and development of an assay that can reliably detect it inside the body is of critical importance.

RoxP (Radical oxygenase of Propionibacterium acnes), is a 16 kDa heme-binding protein, expressed by Cutibacterium genus members. RoxP is an abundantly secreted protein of C. acnes and plays a significant functional role in host-microbe interaction. RoxP aids the viability of monocytes and keratinocytes under oxidative stress conditions. RoxP is remarkably conserved among all phylotypes of C. acnes including several clinical isolates.

Reported herein are four anti-RoxP antibodies, all of which could detect RoxP in indirect ELISA. Taking advantage of the sequence conservation of RoxP among Cutibacterium genus, these antibodies were used to develop a C. acnes specific sandwich ELISA. To verify the utility of this assay in clinically relevant settings, an ELISA based assay was developed that could detect RoxP in several relevant biological fluids. Moreover, 10 different RoxP orthologs were expressed in E. coli and evaluated their binding by individual anti-RoxP antibodies.

### Example 1: Material and Methods

Media and reagents: BH1 (Becton Dickinson) media was prepared by dissolving 37 g powder in 1 L water. ZYM-5052-Carb media was prepared as described previously. Human serum (Thermo Fisher Scientific), Human cerebrospinal fluid (Lee Biosolutions), Human synovial fluid (Innovative Research) and Bovine synovial (Innovative Research) fluid were purchased from commercial vendors.

Antibody generation: RoxP was codon-optimized for E. coli, synthesized, inserted into pET22b for periplasmic expression, produced using autoinduction, NiNTA purified, digested with TEV to remove the His-tag, and then size-exclusion chromatography (SEC) purified. Tagless recombinant RoxP were submitted to GenScript to produce murine anti-RoxP monoclonal antibodies. Twenty hybridoma supernatants were assessed using a biophysical protein binding assay (biolayer-interferometry—BLI) to identify conformational, non-competitive hybridomas. Four non-competitive antibodies were selected. GenScript then prepared 2 mg of each antibody from murine ascites. Biotinylation of antibodies was done with an EZ-LINK™ NHS-PEG4-Biotinylation Kit (Thermo Scientific) according to the manufacturer's instructions.

Indirect ELISA: ELISA 96-well plates were coated with RoxP (1000 ng/ml) in ELISA Coating Buffer (50 mM Carbonate, pH 9.6). The plates were incubated at 4° C. overnight. After three washes with 1×PBST (0.1% Tween-20), the plates were blocked with a blocking solution (1×PBST, 1% BSA) for 1 h at room temperature. After removing the blocking solution, wells were washed 4× with 1×PBST. Then, different concentrations of primary antibodies (4.6-10000 ng/ml for 3E10E3; 2.3-5000 ng/ml for 2H2F3; 1.1-2500 ng/ml for 4D8H1 and 5F3D11) were added to their respective wells and incubated at room temperature for 1 hour. Plates were washed 3× with 1×PBST. After that, plates were incubated with secondary antibody biotin-Goat anti-mIgG (BioLegend) for 1 hour at 37° C. After washing 3× with 1×PBST, Horseradish peroxidase protein A conjugate (GenScript) diluted to 1:5000 was dispensed into each well and incubated at room temperature for 1 hour. After 3 washes with 1×PBST, color was developed by adding TMB reagents (BioLegend) for 30 min. Finally, the optical density (OD) of each well was measured at 450 nm.

For the RoxP ortholog detection experiment, a fixed concentration of primary antibody was used instead of range of concentrations. 2H2F3, 3E10E3, 4D8H1, 5F3D11 antibody concentrations were 330, 2900, 155 and 2100 ng/ml, respectively.

For competition ELISA experiments, serially diluted unlabeled (27-20000 ng/ml) antibodies and b-Abs were added separately at the primary antibody step. Concentrations for b-2H2F3, b-4D8H1 and b-5F3D11 were 500 ng/ml, 250 ng/ml, 250 ng/ml respectively. No secondary antibody was used because detection was based on biotinylated anti-RoxP antibody.

Sandwich ELISA: 96-well plates were coated with capture antibody 4D8H1 (2 μg/ml) in ELISA Coating Buffer (50 mM Carbonate, pH 9.6). The plates were incubated at 4° C. overnight. After three washes with 1×PBST (0.1% Tween-20), the plates were blocked with a blocking solution (1×PBST, 1% BSA) for 1 h at room temperature. After removing the blocking solution, wells were washed 4× with 1×PBST. Then, serially diluted RoxP (0.5-400 ng/ml) were added and incubated at room temperature for 1 hour. Plates were washed 3× with 1×PBST. After that, plates were incubated with the detection antibody b-2H2F3 (250 ng/ml) for 1 hour at RT. After washing 3× with 1×PBST, SA-HRP (GenScript) diluted to 1:5000 or Streptavidin Poly-HRP (Thermo Scientific) was dispensed into each well and incubated at room temperature for 1 hour. After 3 washes with 1×PBST, color was developed by adding TMB reagents (BioLegend) for 30 min. Finally, the optical density (OD) of each well was measured at 450 nm. For the RoxP spike in recovery experiment, 1000 ng/ml RoxP was spiked into serially diluted biofluids and media in the antigen addition step. Everything else was identical to the method described above.

RoxP ortholog selection and multiple sequence alignment: To identify RoxP orthologs, the signal sequence of the reference RoxP (Sequence ID: WP_002515361.1) was identified by SignalP-6.0 and was removed. Then, blastp was used to search the “Non-redundant protein sequences (nr)” database. Ten orthologous sequences were selected based on sequence divergence. A table of selected protein sequence accession numbers and assigned names are provided in Table 1. The multiple sequence alignment of the selected RoxP orthologs was created using MUSCLE and Ugene. The residue background was colored depending on its percentage identity. Solvent accessible surface area values for each residue of RoxP solution structure (PDB: 7BCJ) was calculated using GETAREA.

TABLE 3
Selected protein accession numbers and assigned names.

| | | | SEQ |
|---|---|---|---|
| | | | ID |
| Ortholog name | Accession Number | Assigned Name | NO |
| RoxP_1 | WP_002515361.1 | RoxP_pET22b | 79 |
| RoxP_2 | KPG64580.1 | RoxP_HL201PA1_N31S_T34A_pET22b | 80 |
| RoxP_3 | KFC17752.1 | RoxP_HL201PA1_pET22b | 81 |
| RoxP_4 | WP_070652637.1 | RoxP_HypProt_Alt_1_pET22b | 82 |
| RoxP_5 | WP_002514937.1 | RoxP_HypProt_Alt_2_pET22b | 83 |
| RoxP_6 | WP_098681211.1 | RoxP_HypProt_pET22b | 84 |
| RoxP_7 | WP_042843090.1 | RoxP_Pnam_Alt_1_pET22b | 85 |
| RoxP_8 | WP_063811690.1 | RoxP_Pnam_pET22b | 86 |
| RoxP_9 | WP_002529311.1 | RoxP_S20F_T34P_pET22b | 87 |
| RoxP_10 | AFJ11209.1 | RoxP_T69A_allhorn_pET22b | 88 |
| RoxP_11 | WP_154564006.1 | RoxP_C.porci_pET22b | 89 |
| RoxP_12 | EGG26081.1 | No expression plasmid | 90 |
| RoxP_13 | EGR95586.1 | No expression plasmid | 91 |
| RoxP_14 | OFR52764.1 | No expression plasmid | 92 |
| RoxP_15 | PZA01658.1 | No expression plasmid | 93 |
| RoxP_16 | WP_002524502.1 | No expression plasmid | 94 |
| RoxP_17 | WP_002526392.1 | No expression plasmid | 95 |
| RoxP_18 | WP_002531943.1 | No expression plasmid | 96 |
| RoxP_19 | WP_002532696.1 | No expression plasmid | 97 |
| RoxP_20 | WP_115947204.1 | No expression plasmid | 98 |
| RoxP_21 | WP_230617356.1 | No expression plasmid | 99 |

Expression and purification of recombinant RoxP: For each selected ortholog, signal sequence was removed and an N-terminal leader sequence (MKYLLPTAAAGLLLLAAQPAMAMDHHHHHHHHGGSENLYFQG) (SEQ ID NO: 100) was added that includes a periplasmic secretion signal (PelB: MKY-AMA, amino acids 1-22 of SEQ ID NO: 100), 8His-tag (MDH-HHH (amino acids 23-32 of SEQ ID NO: 100), flexible linker (GGS amino acids 33-35 of SEQ ID NO: 100), and TEV protease site (ENLYFQG, amino acids 36 to 42 of SEQ ID NO: 100). Designed constructs were synthesized by GenScript and cloned into the pET22b expression vector.

RoxP orthologs were expressed by autoinduction where transformed E. coli strains were grown in ZY-5052 media at 16° C. until OD₆₀₀ reached ˜ 10. Cells were collected by centrifuging at 10000 g for 10 minutes. The cell pellet was freeze-thawed once and lysed in B-PER™ Complete Protein Extraction Reagent (Thermo Scientific) for 15 minutes where the following reagents were added to the buffer-Aprotinin (2 ng/ml), Leupeptin (20 nM), Pepstatin A (2 nM), PMSF (1 mM) and EDTA pH 8.0 (1 mM). Lysed cell suspension was cleared by spinning at 16000×g for 20 minutes at 4° C. Then, equal volume of 1×Ni-NTA Buffer A (50 mM Sodium Phosphate, 300 mM Sodium Chloride, 20 mM imidazole) was added to the mixture and His-tagged RoxP was isolated using a Zymo His-Spin Protein Miniprep Kit (Zymo Research) according to manufacturer's instructions. After buffer exchange into PBS using a 2.0 mL Zeba Desalting Column (Thermo Scientific), the mixture was digested with TEV protease (NEB) overnight at 4° C. Then, 1/10 volume of 10×Ni-NTA Buffer base (0.5 M Sodium Phosphate, 3 M Sodium Chloride) was added. The His-tag was removed by collecting the flow-through of a column purification step done using Zymo His-spin columns. Tagless-RoxP was buffer exchanged into HEPES Sizing buffer (20 mM HEPES pH 7.4, 150 mM NaCl, 0.01% azide) using a 2.0 mL Zeba Desalting Columns. Purified proteins were evaluated by SDS-PAGE using AnykD Mini-PROTEAN TGX Precast Gels (BioRad). Protein concentrations were assessed by absorbance at 280 nm using predicted extinction coefficients calculated using ProtParam.

### Example 2: Development of a Highly Sensitive Sandwich ELISA for RoxP Detection

As the first step for developing ELISA-based RoxP detection method, 4 murine anti-RoxP antibodies-2H2F3, 3E10E3, 4D8H1, and 5F3D11 were generated. RoxP (Sequence ID: WP_002515361.1) from the C. acnes strain KPA171202 was selected for antibody generation because it was the first C. acnes strain to be sequenced. Analysis of available RoxP sequences indicated that 32% of C. acnes RoxP shared a 99% identity with this gene.

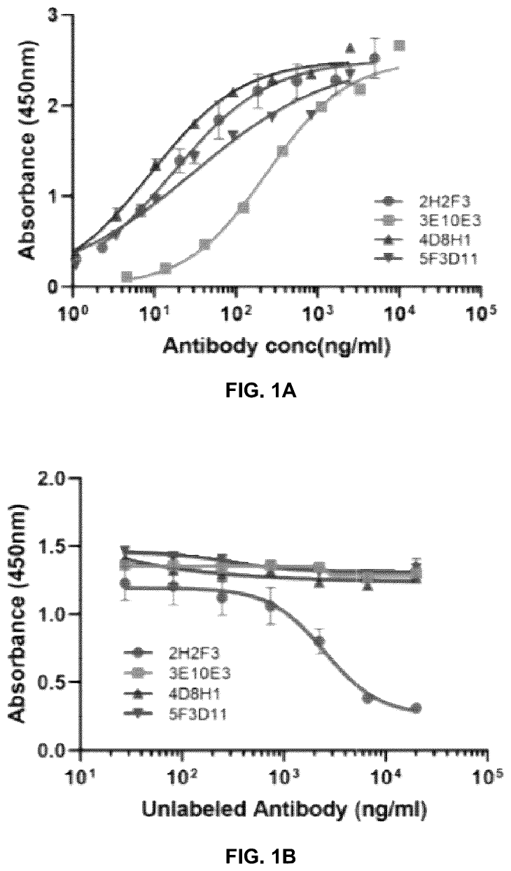

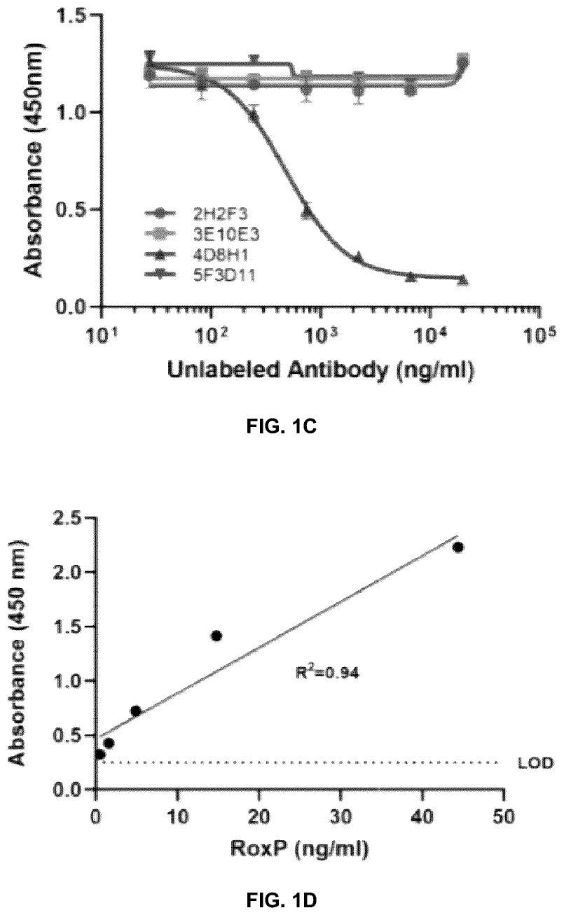

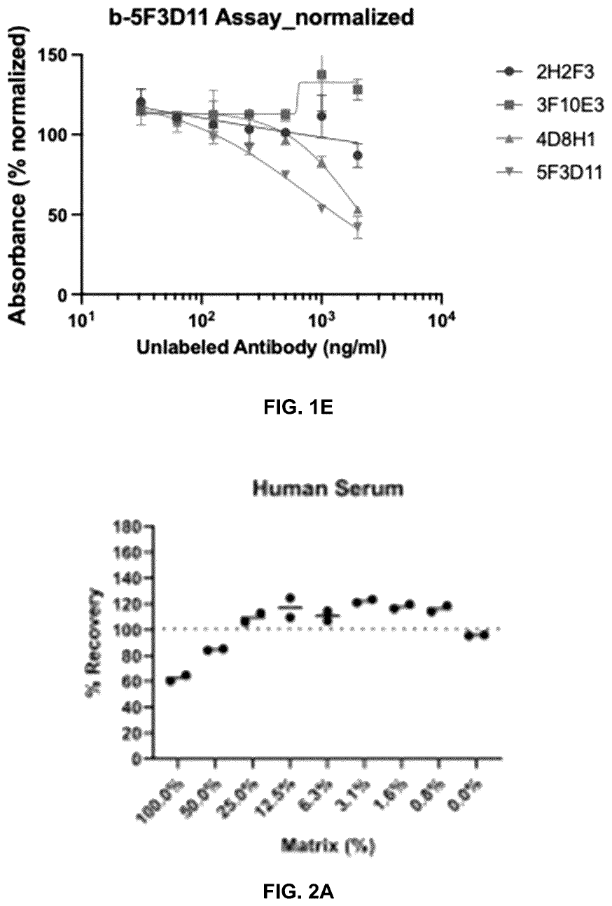

The utility of these antibodies for the detection of RoxP was evaluated by an indirect ELISA (FIG. 1A). When different antibody concentrations were titrated against 1000 ng/ml RoxP, all 4 antibodies were able to detect RoxP. Among them, 4D8H1 was able to detect RoxP at the lowest concentration. Next, all 4 antibodies were biotinylated and their functionality validated by indirect ELISAs (FIG. 1E). Among them, only 3E10E3 was unable to detect RoxP in its biotinylated form, possibly due to biotinylation of a residue that disrupts RoxP binding.

Competition ELISAs were conducted with the biotinylated antibodies (b-2H2F3, b-4D8H1, and b-5F3D11) against a titration of all four unlabeled antibodies. Both b-2H2F3 and b-4D8H1 were well-competed against by their unlabeled counterparts (FIGS. 1B and 1C). Notably, multiple antibodies could compete against 5F3D11 suggesting that binding sites of this antibody may overlap with other antibodies. Consequently, 4D8H1 and 2H2F3 were selected as capture and detection antibodies for sandwich ELISA development. The developed assay was linear up to 50 ng/ml RoxP (FIG. 1D) and could detect RoxP down to 0.5 ng/ml concentration.

### Example 3: Sandwich ELISA can Detect RoxP in Multiple Biofluids

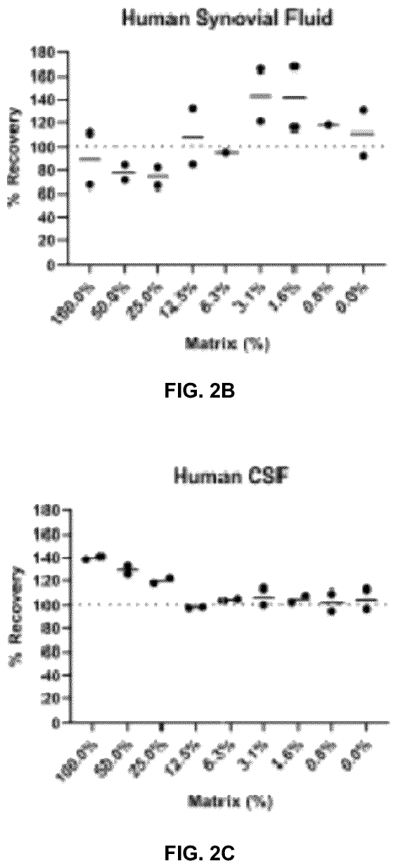

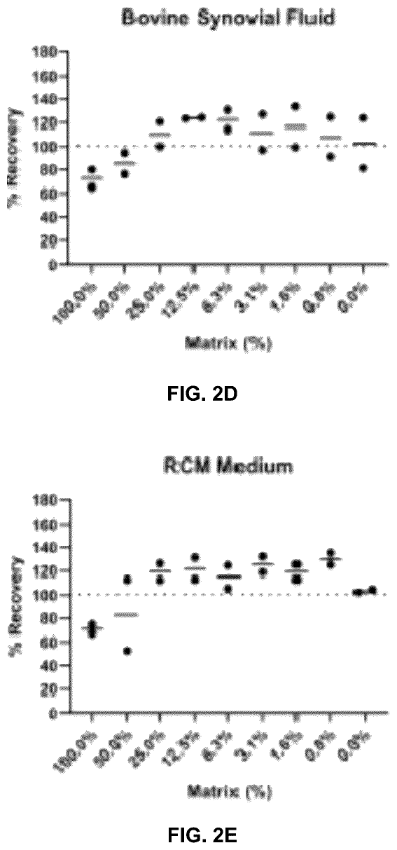

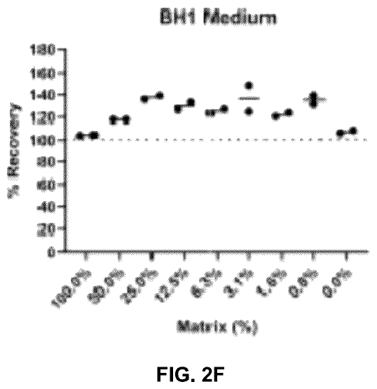

C. acne is primarily found on the head and upper trunk of the body. This explains why C. acnes infections are more commonly observed in these areas after the placement of medical devices, such as neurosurgical shunts, breast implants, heart valve replacements, and prosthetic shoulders. Considering that the sandwich ELISA can be a useful tool for the detection of C. acnes infections, the spike in recovery efficiency of the assay in multiple clinically relevant fluids was evaluated. For this experiment, 50 ng RoxP was spiked into 2× serially diluted human serum, human cerebrospinal fluid, human synovial fluid and bovine synovial fluid. RCM and BH1 media were included in this experiment to broaden the applicability of the assay for research-oriented purposes. Detection of RoxP in Human CSF and BH1 media required no dilution of the biofluids (FIG. 2A-2F). However, all other biofluids required either ¼ or ⅛ dilutions to recover 100% of the spiked in RoxP (FIG. 2A-2F). The assay could detect RoxP in all tested biofluids and media.

### Example 4: ELISA Detection Pattern of RoxP Orthologues Reveal the Antibody Binding Regions

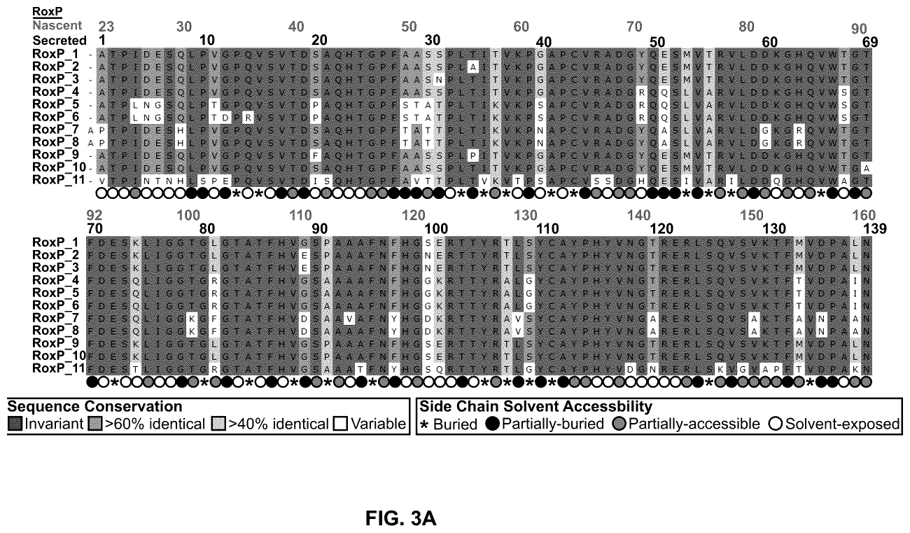

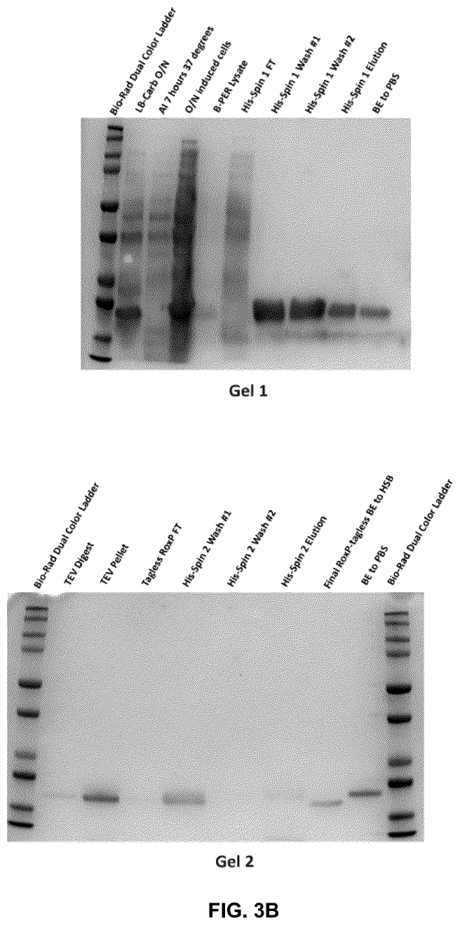

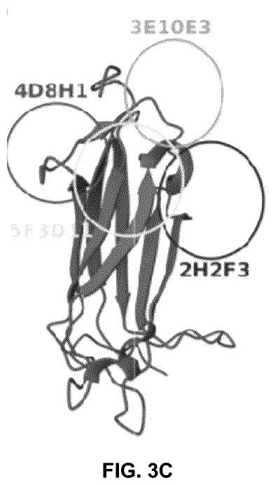

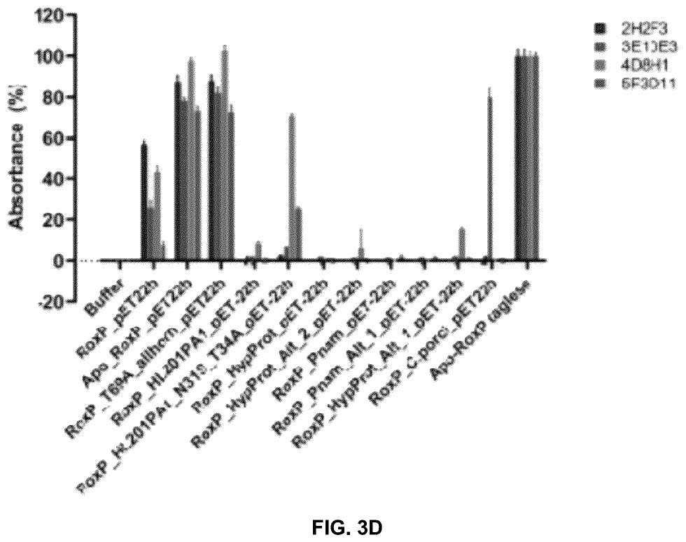

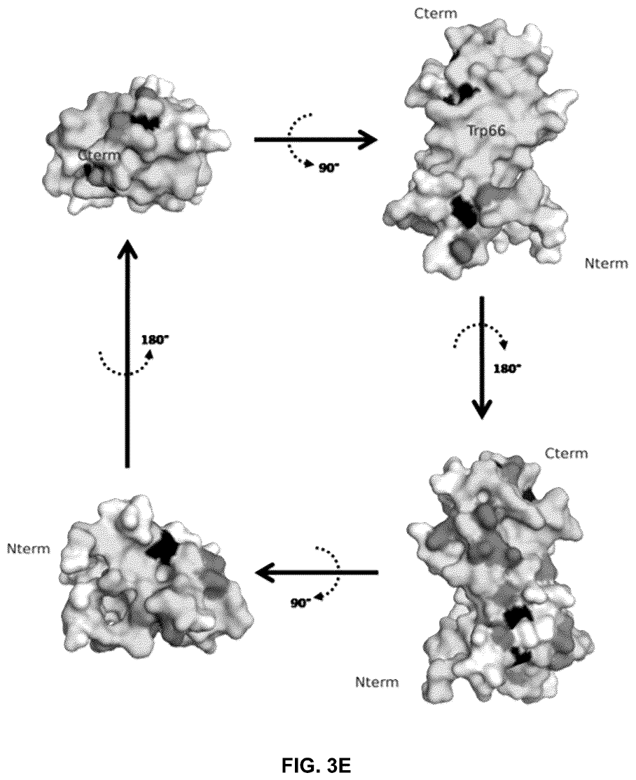

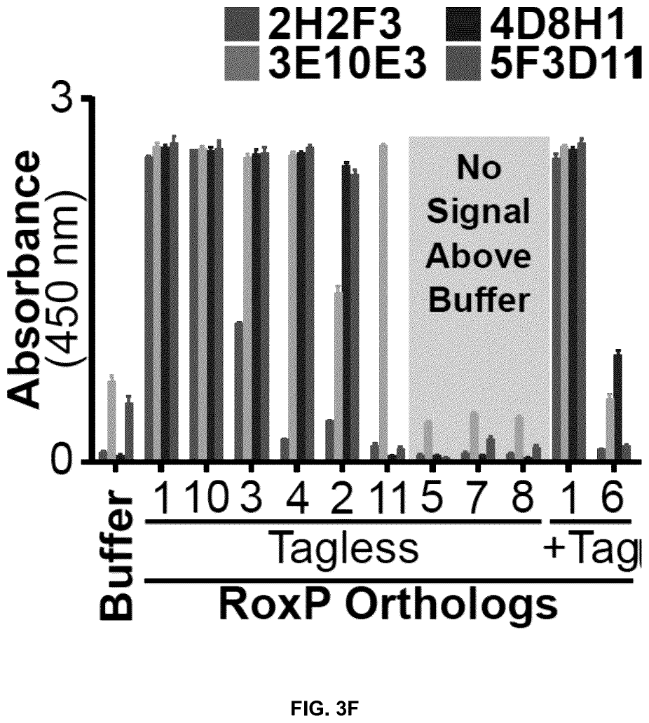

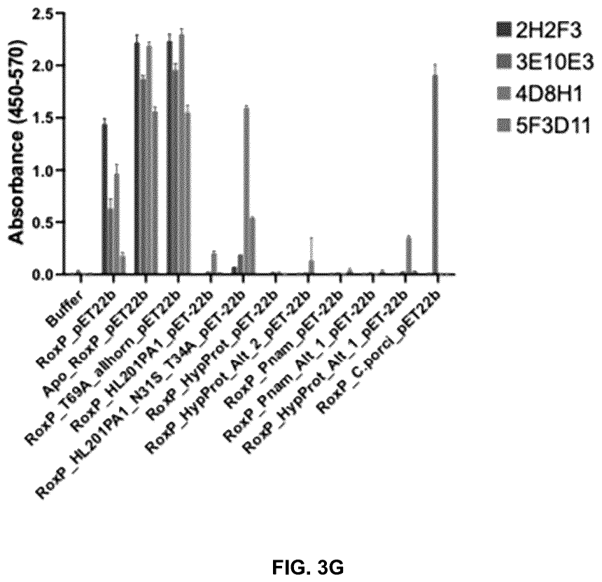

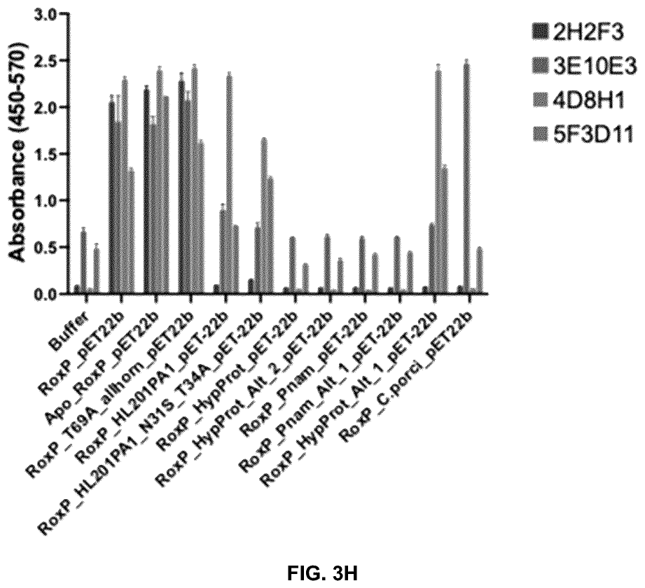

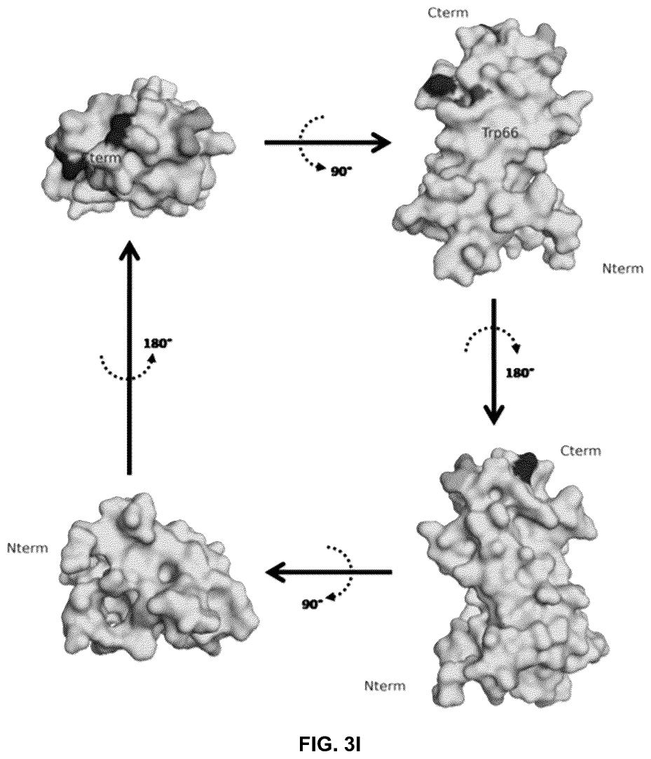

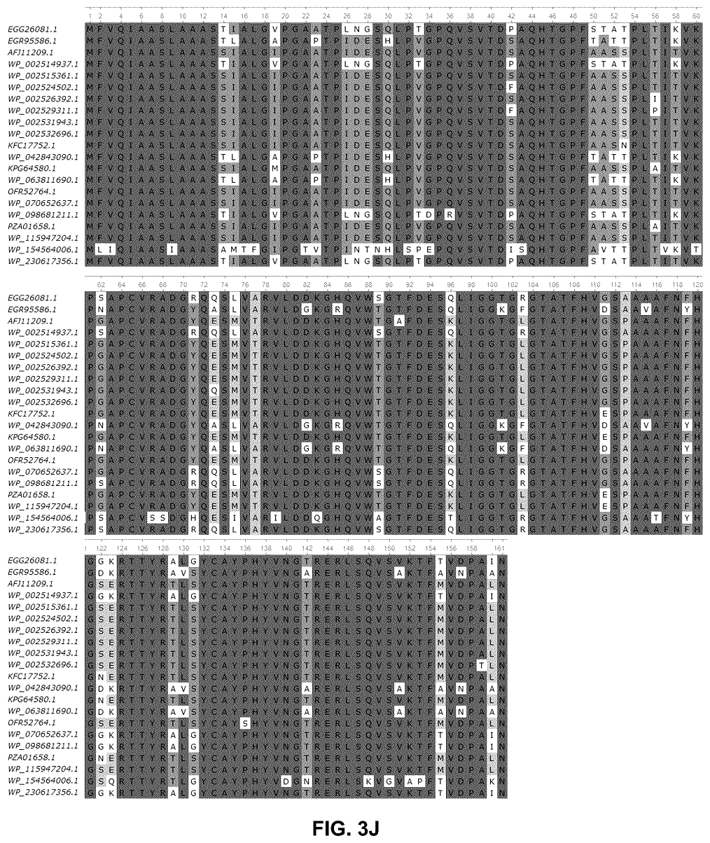

The natural variation among RoxP protein sequences were utilized to evaluate the binding capacity of the anti-RoxP antibodies. For this, reference RoxP protein sequence (RoxP_pET22b) was used as a query for BlastP and 10 RoxP orthologs were selected based on their sequence divergence. Percent sequence identity of the selected orthologs compared to RoxP_pET22b ranged from 99% to 72% accounting for 1 to 39 amino acid residue differences. A multiple sequence alignment of the selected sequences is shown in FIG. 3A and FIG. 3J. Though RoxP is highly conserved, analysis of surface exposure of RoxP non-conservative amino acids indicated that sequence variation does exist in potentially important positions on the surface (FIG. 3E). A HIS-tagged version of each of the orthologs was expressed using a pET22b vector-based expression system. Each ortholog was purified while monitoring the purification process by SDS-PAGE. An example gel image for the purification steps is shown in the FIG. 3B. Both RoxP_S20F_T34P_pET22b and RoxP_HypProt_pET-22b were found to be insoluble during the purification process. However, RoxP_HypProt_pET-22b became insoluble only after removal of the HIS-tag. So, this protein was purified without HIS-tag removal and to enable its direct comparison with reference RoxP, a HIS-tagged version of RoxP_pET22b R (i.e. Apo_RoxP_pET22b) was purified. All other orthologs were soluble after HIS-tag removal.

Recombinant RoxP orthologs were then evaluated for detection by 2H2F3, 3E10E3, 4D8H1, and 5F3D11 antibodies (FIG. 3C-3D, FIG. 3F-3H). Generally, RoxP orthologs that less divergent (i.e. 1-3 amino acids difference) compared to the reference RoxP_pET22b showed better binding. For example, RoxP_T69A_allhorn_pET22b, which is only 1 amino acid different compared to RoxP_pET22b, exhibited a similar binding pattern like RoxP_pET22b. But, as the protein sequences diverged more, the success rate of detection decreased for the anti-RoxP antibodies. This gave a unique opportunity to predict the binding regions of anti-RoxP antibodies by comparing their binding to RoxP orthologs. Recently, RoxP solution structure by NMR spectroscopy has been published. The predicted antibody binding regions were juxtaposed on this structure and shown in FIG. 3C and FIG. 3I.

### Example 4: Discussion

Herein, is reported a highly sensitive ELISA-based method for the detection of C. acnes protein RoxP. The ELISA assay can recover spiked in RoxP from multiple biofluids and media. Additionally, 10 orthologs of RoxP were expressed and their anti-RoxP antibody binding patterns that shed light on the binding regions of these antibodies.

Till date, there are no medical interventions available that specifically target C. acnes infections. So, new strategies to eliminate surgical site C. acnes are needed to prevent disease and promote longevity. In this study, four anti-RoxP antibodies-2H2F3, 3E10E3, 4D8H1 and 5F3D11 were generated. Each of these antibodies can bind RoxP as shown in our indirect ELISA (FIG. 1A) and has distinct binding regions (FIG. 3C). RoxP is required for the survival of C. acnes in aerobic conditions. It is also vital for skin colonization of C. acnes. The antibodies target a universally secreted and cell-surface associated protein (RoxP) that has the better chance to improve clinical outcomes compared to previously published antibodies.

According to a BLAST analysis, besides C. acnes, there are no other bacteria that encode RoxP, except for the closely related Propionibacterium humerusii. One of the selected RoxP ortholog RoxP_HypProt_Alt_2_pET22b (Accession number WP_002514937.1) represents a RoxP sequence from Cutibacterium modestum, which is the same species as P. humerusii. The indirect ELISA experiment (FIG. 3D) showed that the 2H2F3 antibody does not bind RoxP_HypProt_Alt_2_pET22b, whereas the 4D8H1 antibody exhibited a low binding signal. Since 4D8H1-2H2F3 antibody pairs were used in our sandwich ELISA we assume that this assay is C. acnes specific.

Recently, the molecular imprinting technique has been employed to create capacitive and surface plasmon resonance biosensors for RoxP detection. Between these two, the surface plasmon resonance biosensor generated more consistent results and was able to detect RoxP at a concentration as low as 0.23 nM. Even though this assay for RoxP is highly sensitive, the underlying technique is currently not available in clinical practice. In comparison, the sandwich ELISA can be converted to a laminar flow assay and implemented in a clinical setting as a simple point-of-care assay for C. acnes infection detection.

In summary, a sandwich ELISA was developed to detect RoxP from the opportunistic pathogen Cutibacterium acnes. The assay has high sensitivity and precision, and can be implemented in a clinical settings which will reduce both the time and cost of treatment for C. acnes infected patients.

What is claimed is:

1. An isolated antibody, epitope binding fragment, or variant thereof that binds to Rox-P, comprising:
(a) an immunoglobulin heavy chain variable region (VH) at least 90% identical to the amino acid sequence set forth in one of SEQ ID NOS: 1-4; or
(b) an immunoglobulin light chain variable region (VL) at least 90% identical to the amino acid sequence set forth in one of SEQ ID NOS: 5-8; or
(c) both (a) and (b) above.

2. The isolated antibody, epitope binding fragment or variant thereof of claim 1, comprising one or more of:
(a) a HC-CDR1 comprising an amino acid sequence at least 80% identical to the amino acid sequence of SEQ ID NO: 9, 15, 21, or 27;
(b) a HC-CDR2 comprising an amino acid sequence at least 80% identical to the amino acid sequence of SEQ ID NO: 10, 16, 22, or 28;
(c) a HC-CDR3 comprising an amino acid sequence at least 80% identical to the amino acid sequence of SEQ ID NO: 11, 17, 23, or 29;
(d) a LC-CDR1 comprising an amino acid sequence at least 80% identical to the amino acid sequence of SEQ ID NO: 12, 18, 24, or 30;
(e) a LC-CDR2 comprising an amino acid sequence at least 80% identical to the amino acid sequence of SEQ ID NO: 13, 19, 25, or 31; or
(f) a LC-CDR3 comprising an amino acid sequence at least 80% identical to the amino acid sequence of SEQ ID NO: 14, 20, 26, or 32.

3. The isolated antibody, epitope binding fragment, or variant thereof of claim 1, wherein the VH comprises an amino acid sequence at least 90% identical to the amino acid sequence set forth in any one of SEQ ID NOs: 1-4.

4. The isolated antibody, epitope binding fragment, or variant thereof of claim 1, wherein the VL comprises an amino acid sequence at least 90% identical to the amino acid sequence set forth in any one of SEQ ID NOs: 5-8.

5. The isolated antibody, epitope binding fragment, or variant thereof of claim 1, wherein the antibody comprises:
a heavy chain variable region having the sequence of SEQ ID NO. 1 and a light chain variable region having the sequence of SEQ ID NO. 5;
a heavy chain variable region having the sequence of SEQ ID NO. 1 and a light chain variable region having the sequence of SEQ ID NO. 6;
a heavy chain variable region having the sequence of SEQ ID NO. 1 and a light chain variable region having the sequence of SEQ ID NO. 7;
a heavy chain variable region having the sequence of SEQ ID NO. 1 and a light chain variable region having the sequence of SEQ ID NO. 8;
a heavy chain variable region having the sequence of SEQ ID NO. 2 and a light chain variable region having the sequence of SEQ ID NO. 5;
a heavy chain variable region having the sequence of SEQ ID NO. 2 and a light chain variable region having the sequence of SEQ ID NO. 6;
a heavy chain variable region having the sequence of SEQ ID NO. 2 and a light chain variable region having the sequence of SEQ ID NO. 7;
a heavy chain variable region having the sequence of SEQ ID NO. 2 and a light chain variable region having the sequence of SEQ ID NO. 8;
a heavy chain variable region having the sequence of SEQ ID NO. 3 and a light chain variable region having the sequence of SEQ ID NO. 5;
a heavy chain variable region having the sequence of SEQ ID NO. 3 and a light chain variable region having the sequence of SEQ ID NO. 6;
a heavy chain variable region having the sequence of SEQ ID NO. 3 and a light chain variable region having the sequence of SEQ ID NO. 7;
a heavy chain variable region having the sequence of SEQ ID NO. 3 and a light chain variable region having the sequence of SEQ ID NO. 8;
a heavy chain variable region having the sequence of SEQ ID NO. 4 and a light chain variable region having the sequence of SEQ ID NO. 5;
a heavy chain variable region having the sequence of SEQ ID NO. 4 and a light chain variable region having the sequence of SEQ ID NO. 6;
a heavy chain variable region having the sequence of SEQ ID NO. 5 and a light chain variable region having the sequence of SEQ ID NO. 7; or
a heavy chain variable region having the sequence of SEQ ID NO. 6 and a light chain variable region having the sequence of SEQ ID NO. 8.

6. The isolated antibody, epitope binding fragment, or variant thereof of claim 1, wherein the antibody is a full-length antibody.

7. The isolated antibody, epitope binding fragment, or variant thereof of claim 6, wherein the antibody is any one of a monoclonal antibody, an IgG, Fv, single chain antibody, nanobody, diabody, scFv, Fab, F(ab′) 2, and Fab.

8. The isolated antibody, epitope binding fragment or variant thereof of claim 1, further comprising a signal sequence.

9. The isolated antibody, epitope binding fragment, or variant thereof of claim 1, further comprising a detection molecule.

10. The isolated antibody, epitope binding fragment, or variant thereof of claim 9, wherein the detection molecule comprises a fluorescent label, phosphorescent molecule, chemiluminescent molecule, radioactive isotope, chromophore, luminescent molecule, photoaffinity molecule, colored particle and/or ligand.

11. A polynucleotide sequence encoding an isolated antibody, epitope binding fragment or variant thereof of claim 1.

12. The polynucleotide of claim 11, wherein the polynucleotide comprises a nucleic acid sequence at least 80% identical to:
(a) a nHC-CDR1 corresponding to any one of SEQ ID NO: 41, 47, 53, or 59;
(b) a nHC-CDR2 corresponding to any one of SEQ ID NO: 42, 48, 54, or 60;
(c) a nHC-CDR3 corresponding to any one of SEQ ID NO: 43, 49, 55, or 61;
(d) a nLC-CDR1 corresponding to any one of SEQ ID NO: 44, 50, 56, or 62;
(e) a nLC-CDR2 corresponding to any one of SEQ ID NO: 45, 51, 57, or 63; or
(f) a nLC-CDR3 corresponding to any one of SEQ ID NO: 46, 52, 58, or 64.

13. The polynucleotide of claim 12, encoding a variable heavy chain (VH) comprising a nucleic acid sequence at least 90% identical to any one of SEQ ID NOs: 33-36.

14. The polynucleotide of claim 12, encoding a variable light chain (VL) comprising a nucleic acid sequence at least 80% identical to one of SEQ ID NOs: 37-40.

15. A method of determining an amount of Cutibacterium sp in a sample, the method comprising:
(a) contacting the sample with the isolated antibody, epitope binding fragment or variant thereof of claim 1; wherein the isolated antibody, epitope binding fragment or variant thereof specifically binds to RoxP; to form an antibody-RoxP complex;
(b) using a detector to determine presence of the antibody-RoxP complex;
(c) quantifying the levels of RoxP in the antibody-RoxP complex; and
(d) determining the amount of Cutibacterium sp in the sample based on the quantity of RoxP in step c.

16. The method of claim 15, wherein the isolated antibody, epitope binding fragment, or variant thereof further comprises a detection molecule selected from a fluorescent label, a phosphorescent molecule, a chemiluminescent molecule, a radioactive isotope, a chromophore, a luminescent molecule, a photoaffinity molecule, a colored particle, and a ligand.

17. The method of claim 15, further comprising contacting the antibody-RoxP complex with a reporter molecule that specifically binds to the isolated antibody, epitope binding fragment or variant thereof and/or the antibody-RoxP complex, wherein the reporter molecule comprises a detector molecule.

18. The method of claim 17, wherein the reporter molecule is a secondary antibody.

19. The method of claim 15, wherein the sample is a biological sample from a subject, wherein the biological sample is selected from a biological fluid, tissue, and cell culture.

20. The method of claim 19, wherein the biological fluid is selected from CSF, blood, saliva, tears, and pus.
